(19) United States
(12) Patent Application Publication
(10) Pub. No.: US 2006/0183888 A1
(43) Pub. Date: Aug. 17, 2006

(54) Pyrrolysine synthesis and modification

(76) Inventors: Michael K. Chan, Hilliard, OH (US); Joseph A. Krzycki, Columbus, OH (US)

Correspondence Address: CALFEE HALTER & GRISWOLD, LLP, 800 SUPERIOR AVENUE, SUITE 1400, CLEVELAND, OH, 44114, US

(21) Appl. No.: 11/203,820

(22) Filed: Aug. 15, 2005

Related U.S. Application Data
(60) Provisional application No. 60/601,228, filed on Aug. 13, 2004.

(51) Int. Cl. C07K 14/47; C07D 207/12

(52) U.S. Cl. 530/409; 548/537; 548/303.7

(57) ABSTRACT

Synthetic L-pyrrolysine of formula I, and derivatives thereof, chemical methods of preparing these amino acids and derivatives, and methods of derivatizing these amino acids after incorporation into a peptide or protein. 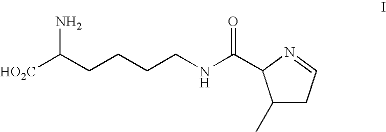 Also provided are derivatives and methods of preparing derivatives, including addition of substituents at any substitutable position; modifications to the lysine alkyl chain; modifications of the pyrroline ring. Also provided are methods of adding labels to the pyrrolysine or derivative thereof.

### CROSS-REFERENCE TO RELATED APPLICATIONS

This application claims priority to U.S. Provisional Patent Application No. 60/601,228; filed Aug. 13, 2004, entitled “Charging tRNA with Pyrrolysine, the entirety of which is incorporated herein by reference.

### STATEMENT REGARDING FEDERALLY FUNDED RESEARCH

This work was supported at least in part by grants from the National Science Foundation (MCB-9808914); the Department of Energy (DE-FG02-92ER20042); and the National Institutes of Health (GM 061796). The Federal Government may have certain rights in this invention.

### BACKGROUND OF THE INVENTION

A need exists for methods of labeling proteins, and specifically the ability to label proteins at a particular location. Additionally, it would be highly desirable to have the ability to be able to not only label proteins at specific locations, but also with a number of different possible labels.

### SUMMARY OF THE INVENTION

Provided herein is a synthetic amino acid having the structure:

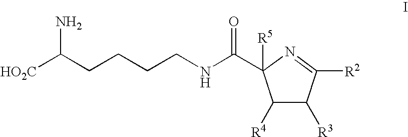

wherein R², R³, R⁴, R⁵ are selected from H, alkyl, halide, hydroxyl, amino, thiol, phosphoryl, azido, alkynyl, aldehyde, carboxylate, ester, a fluorescent label, affinity tag, a nucleic acid binding group, a latent chemical crosslinking group, a photoactivatable crosslinking group and combinations thereof; and salts thereof. In some embodiments, the amino acid or derivative thereof is an L-pyrrolysine or L-pyrrolysine derivative. In other embodiments, the chirality may be altered at any stereocenter. In some embodiments, R², R³, R⁴, and R⁵ further contain a substituent selected from a fluorescence tag, a spin tag, a binding agent, or combinations thereof. In some embodiments, the synthetic amino acid or derivative may contain a radioactive element.

Provided in another embodiment is the pyrrolysine derivative of formula II:

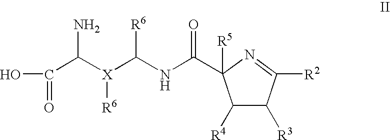

In some embodiments, the heteroalkyl is selected from ether, thioether, dialkylamine and (CₙH₂ₙX′CₙH₂ₙY′CₘH₂ₘ); wherein m and n may be the same or different and may be 0-6; X′ and Y′ are selected from CR₇R₈, SiR₇R₈, O, S, Se, Te, NR₇, PR₇, AsR₇ and R₇ and R₈ are alkyl or heteroalkyl, and which may further be substituted with H, alkyl chain, halide, hydroxyl, amino, thiol, phosphoryl, azido, alkynyl, aldehyde, carboxylate, ester, a fluorescent label, affinity tag, a nucleic acid binding group, a latent chemical crosslinking group, a photoactivatable crosslinking group and combinations thereof. In some embodiments, the compounds of formula II may include a radioactive element.

In some embodiments, the heteroalkyl is selected from ether, thioether, dialkylamine and (CₙH₂ₙX′CₙH₂ₙY′CₙH₂ₙ); wherein X′ and Y′ are selected from CR₇R₈, SiR₇R₈, O, S, Se, Te, NR₇, PR₇, AsR₇ and R₇ and R₈ are alkyl or heteroalkyl, and which may further be substituted with H, alkyl chain, halide, hydroxyl, amino, thiol, phosphoryl, azido, alkynyl, aldehyde, carboxylate, ester, a fluorescent label, affinity tag, a nucleic acid binding group, a latent chemical crosslinking group, a photoactivatable crosslinking group and combinations thereof. In some embodiments, the compounds of formula II may include a radioactive element.

In another embodiment, the pyrrolysine derivative is that of formula III:

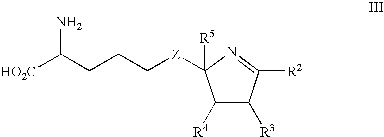

wherein Z is selected from ester, ketone, ether, amido, thioether, and amide; wherein if the linkage is amide, the amide carbonyl and nitrogen are exchanged relative to their positions in pyrrolysine; and wherein R², R³, R⁴, R⁵ are selected from a proton, alkyl chain, halide, hydroxyl, amino, thiol, phosphoryl, azido, alkynyl, aldehyde, carboxylate, ester, a fluorescent label, affinity tag, a nucleic acid binding group, a latent chemical crosslinking group, a photoactivatable crosslinking group and combinations thereof; or a salt thereof. In some embodiment, one or more of R², R³, R⁴, and R⁵ may further have a substituent selected from a fluorescence tag, a spin tag, a binding agent, a latent chemical crosslinking group, a photoactivatable crosslinking group and combinations thereof. In still other embodiments, the amino acid derivative may contain a radioactive element.

In some specific embodiments, the derivative may be one of the following

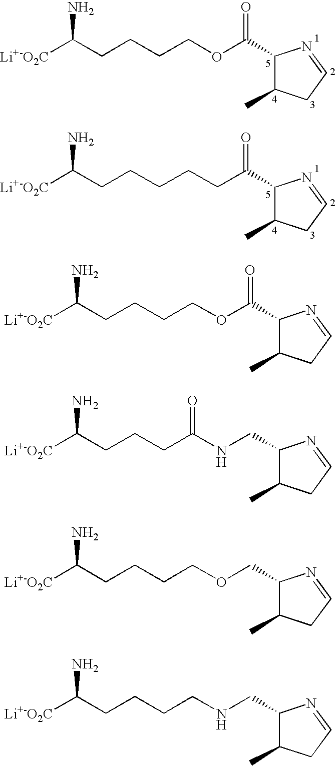

or a salt thereof.

These derivatives may have more substituents on any substitutable C atom on the lysine chain, the substituent selected from alkyl, substituted alkyl, alkynyl, and substituted alkynyl, halide, hydroxyl, amino, thiol, aldehyde, carboxylate, ester, wherein the substituent on the substituted alkyl or alkynyl is selected from azido, fluorescence tag, spin tag, specific binding agent, a latent chemical crosslinking group, a photoactivatable crosslinking group and combinations thereof. In some embodiments, these derivatives may also have one or more substituents on the carbon atoms of the pyrrole ring, the substituents selected from alkyl chain, halide, hydroxyl, amino, thiol, phosphoryl, azido, alkynyl, aldehyde, carboxylate, ester, a fluorescent label, affinity tag, a nucleic acid binding group, a latent chemical crosslinking group, a photoactivatable crosslinking group and combinations thereof.

In another embodiment, the pyrrolysine derivative is that of formula IV:

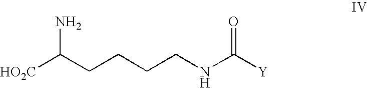

wherein Y is selected from cycloalkyl, heterocycloalkyl, biotin derivative, 7-dimethylaminocoumarin-4-acetic acid and 7-hydroxycoumarin-3-carboxylic acid succinimidyl ester; and wherein Y is cycloalkyl or heterocycloalkyl the carbon atoms of the cycloalkyl or heterocycloalkyl have substituents selected from H, alkyl, halide, hydroxyl, amino, thiol, phosphoryl, a fluorescent label, a spin label, an affinity tag, a nucleic acid binding group, a latent chemical crosslinking group, a photoactivatable crosslinking group; or a salt thereof.

In some embodiments of formula IV, Y may be selected from 3-membered rings, 4-membered rings, 5-membered ring, and 6-membered rings; and wherein the 3-, 4-, 5-, or 6-membered rings may have one or two N's, one or two O's or one N and one O in the ring, and wherein the rings may have one or more unsaturated bonds. Some specific embodiments include, but are not limited to those wherein Y is pyrroline, pyrroline in its enamine form, proline, cyclopentane, hydrofuran, thiophene, cyclopropane, and aziridine. In some embodiments, the compounds of formula IV may include a radioactive element.

Further provided is a A method for the synthesis of L-pyrrolysine, the method having the the steps of: a) preparing 4-methyl-substituted glutamate γ-semialdehyde; and b) cyclizing 4-methyl-substituted glutamate γ-semialdehyde to form the desired pyrrole ring.

Also provided is a method for the synthesis of L-pyrrolysine or a derivative thereof wherein the method includes the step of coupling of a carboxylate group or derivative thereof to the epsilon N of lysine or a lysine analog.

Also provided is a method for the chemical addition of functional groups to pyrrolysine or a derivative thereof following incorporation into recombinant protein, the method having the steps of: a) incorporating pyrrolysine or a derivative thereof into a recombinant protein; and b) adding an activated form of a modifying group selected from fluorescent labels, spin labels, affinity tags, nucleic acid binding groups, photolabile groups or radioactive elements.

Also provided is a method for the chemical modification of pyrrolysine or a derivative thereof by reacting the pyrrolysine or derivative with a reactive group selected from a reducing group, a nucleophile, an electrophile, and an oxidizing group.

### BRIEF DESCRIPTION OF THE DRAWINGS

FIG. 1 shows the alignment of PylS sequences from Methanosarcinacea PylS examples. Top to bottom are Methanosarcina mazei (mazpylS) SEQ ID NO: 7, Methanosarcina acetivorans (acetPylS) SEQ ID NO: 8, Methanosarcina barkeri MS (MSPylS), SEQ ID NO: 9, Methanosarcina barkeri Fusaro (fusPylS) SEQ ID NO: 10, and Methanococcoides burtonii (PylSMccoides) SEQ ID NO: 11. The Desulfitobacterium hafniense PylS gene is split into two genes, pylSn, and pylSc, and encodes 2 gene products SEQ ID NO:6 and SEQ ID NO:12. Both the PylSn and PylSc gene products are homologous to the methanogen PylS proteins, and together are likely to charge tRNA^{Pyl} with pyrrolysine or other derivatives.

FIG. 2 shows alignment of the catalytic core (SEQ ID NOs 1-5) of PylS examples of the methanogenic Arachaea shown in FIG. 1. The predicted product of the pylSc gene (SEQ ID NO: 6) from Desulfitobacterium hafniense is also shown. Also shown is a consensus sequence, SEQ ID NO: 13.

FIG. 3 shows three motifs in the catalytic core of the pyrrolysyl tRNA syntetases examples shown in FIG. 1.

FIG. 4 shows the secondary structure and sequences of three representative examples of pylT gene product, tRNA^{Pyl} (also known as tRNA_{CUA}). From left to right are shown the tRNA^{Pyl} from D. hafniense, Mc. burtoniii, and Methanosarcina spp. Methanosarcina spp. include Methanosarcina barkeri Fusaro, Methanosarcina mazei, Methanosarcina acetivorans (which all have identical tRNA^{Pyl}), as well as Methanosarcina barkeri MS (which has the substitutions indicated from the other Methanosarcina spp.) The boxes indicate bases that are deviated from the Methanosarcina spp. structure typlified by M. barkeri Fusaro. Circles on the Methanosarcina spp. indicate bases that are conserved in all known examples of tRNA^{Pyl}.

FIG. 5 is an alignment of the conserved regions in the N terminal domain of the pyrrolysyl-tRNA synthetases from the five methanogenic Archaea, SEQ ID NOs. 26-30, a corresponding sequence, SEQ ID NO: 31, from the PlySn gene product of D. hafneinse and a consensus sequence, SEQ ID NO: 25, derived from this alignment.

### DETAILED DESCRIPTION OF THE INVENTION

The present invention will now be described by reference to more detailed embodimehts, with occasional reference to the accompanying drawings. This invention may, however, be embodied in different forms and should not be construed as limited to the embodiments set forth herein. Rather these embodiments are provided so that this disclosure will be thorough and complete, and will convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

All publications, patent applications, patents, and other references mentioned herein are incorporated by reference in their entirety.

Definitions

The term “purified” as used herein does not require absolute purity; rather, it is intended as a relative term. Thus, for example, a purified amino acid is one in which the amino acid is more enriched than the amino acid is in its natural environment within a cell. Preferably, a preparation is purified such that the amino acid represents at least 50% of the amino acid content of the preparation.

By a “pyrrolysyl tRNA synthetase polypeptide” is meant a polypeptide having pyrrolysl tRNA synthetase biological activity. The PylT gene product described herein may be referred to as tRNA_{CUA} or as tRNA^{pyl}; these two terms are used interchangeably.

“Promoter,” as used herein, refers to sequences in DNA which mediate initiation of transcription by an RNA polymerase. Transcriptional promoters may comprise one or more of a number of different sequence elements as follows: 1) sequence elements present at the site of transcription initiation; 2) sequence elements present upstream of the transcription initiation site and; 3) sequence elements downstream of the transcription initiation site. The individual sequence elements function as sites on the DNA where RNA polymerases, and transcription factors that facilitate positioning of RNA polymerases on the DNA, bind.

Methods of Preparing Proteins Comprising Pyyrolysine and Pyrrolysine Derivatives.

Most organisms employ UAG as a stop codon, but translation is not terminated at in-frame UAGs in some methyltransferases of methanogenic Archaea. Rather, these codons serve as sense codons and, as determined by crystal structure analyses, UAG encodes pyrrolysine, (4R, 5R)-4-substituted-pyrroline-5-carboxylate, the 22nd amino acid found to be genetically encoded in nature. A key question was whether the UAG-translating tRNA_{CUA} is first charged with lysine and then modified to pyrrolysine for incorporation into the growing polypeptide or whether pyrrolysine is attached as the fully synthesized amino acid to tRNA_{CUA}. We have found that the latter possibility is feasible by demonstrating the direct pyrrolysylation of tRNA_{CUA} in vitro. This is the first example found in nature of specific aminoacylation of a tRNA with a non-canonical amino acid. The results reported show further that the expression of only two genes, pylT and pylS, that encode tRNA_{CUA} and pyrrolysyl-tRNA synthetase, can expand the genetic code of E. coli to include pyrrolysine. This procedure could potentially be used to immediately expand the genetic code of any species that can incorporate exogenously added pyrrolysine.

The present invention encompasses methods of preparing modified proteins comprising a pyrrolysine residue, methods of preparing modified cells that produce proteins comprising a pyrrolysine residue, and the modified cells that are produced in accordance with such methods. Also included, are kits for introducing a pyrrolysine into protein or polypeptide encoded by a polynucleotide comprising an in-frame UAG or TAG codon.

Preparation of Modified Cells that Produce Proteins Comprising a Pyrrolysine Residue.

In one aspect, the present invention provides a method of preparing a modified cell that, when exposed to pyrrolysine and an mRNA comprising an in-frame UAG or TAG codon, incorporates a pyrrolysine residue into the protein or polypeptide encoded by the mRNA. The method comprises the steps of providing an unmodified cell that lacks a product of the pyrrolysyl-tRNA synthetase gene, or a transfer RNA comprising a CUA anticodon (tRNA^{Pyl}), or both; incorporating an expression construct comprising a polynucleotide that encodes a Group I or Group II pyrrolysyl-tRNA synthetase and an expression construct comprising a polynucleotide encoding a pyrrolysine transfer RNA into the cell; and maintaining the cell under conditions that permit expression of the pyrrolysyl-tRNA synthetase and the tRNA for pyrrolysine.

Pyrrolysyl-tRNA Synthetase

Pyrrolysyl-tRNA Synthetase (PylS) is an enzyme that is capable of two reactions required to charge tRNA with pyrrolysine.

Formation of the pyrrolysyl-adenylate and pyrophosphate from pyrrolysine and ATP, measured by PPi:ATP exchange reaction dependent on pyrrolysine. This activates the amino acid for tRNA charging.

Specific charging of tRNA^{Pyl} (also known as tRNA_{CUA}) with pyrrolysine in the presence of ATP. This is measured by an acid denaturing gel shift dependent on reactants and enzyme.

PylS is the first non-canonical aminoacyl-tRNA synthetase to be found in nature. PylS can fall into two groups, Group I (represented by M. barkeri Fusaro) and Group II (sole known representative is PylS from Desulfitobacterium haniense). Group I enzymes are encoded as single genes that give rise to proteins with monomeric molecular weight (MW) of 50 kDa. Their relative identities average 79% and above. The group II enzyme is encoded by two separate genes, the pylSn gene and the pylSc genes. The pylSn gene has low identity with the N-terminal domain of class I PylS enzymes. However, the pylSc gene encodes a protein with 45% identity (64% similarity) to the C-terminal domain (catalytic domain) of class I PylS enzymes. In both the methanogenic Archea and the gram positive bacterium D. hafniense, Group I and Group II PylS are encoded by pylS genes associated with the pylT, pylB, pylC, and pylD genes. (See Figure A) The pyT gene encodes an UAG-decoding tRNA with unusual properties. The association of pylS from both groups with pylT indicates a common functionality for PylS in charging tRNA^{Pyl} with pyrrolysine.

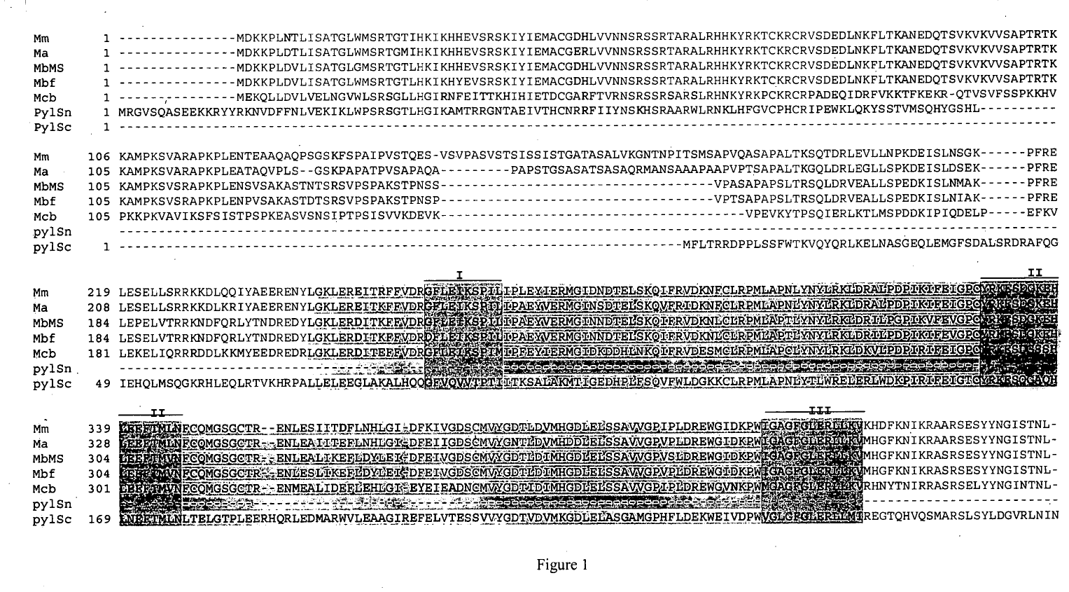

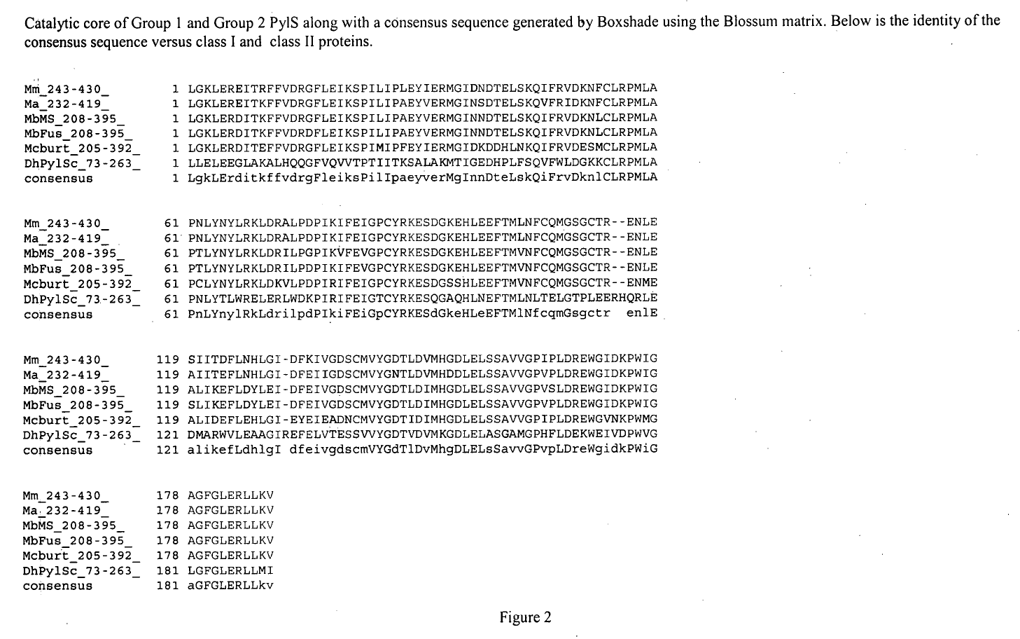

FIG. 1 depicts a global alignment of the complete sequences of the known Group 1 and Group 2 PylS enzymes. It shows the “catalytic core” of PylS as blue and gray shaded sequence encompassing the three motifs characteristic of class 2 aminoacyl-tRNA synthetases. FIG. 2 depicts an alignment of this same catalytic core of five known Group I PyIS sequences from the methanogenic archea Methanosarcina mazei (Mm) (SEQ ID NO: 1), Methanosarcina acetivorans (Ma), SEQ ID NO: 2, Methanosarcina barkeri MS (MbMS) SEQ ID NO: 3, Methanosarcina barkeri Fusaro (MbFus) SEQ ID NO: 4, and Methanococcoides burtonii (Mcburt) SEQ ID NO: 5, and one Group II PylSc sequence (SEQ ID NO: 6) from the gram positive bacterium Desulfitobacterium haniense. FIG. 2 also depicts a consensus sequence, SEQ ID NO: 13, generated by Boxshade using the Blosum matrix. The alignment itself was generated with Clustal 1.8 using the Blosum matrix.

The aligned pylS sequences are

Group 1 PylS sequences:

Methanosarcina mazei Go1,gi|20905927|gb|AAM31141.1|,

SEQ ID NO: 7

MDKKPLNTLISATGLWMSRTGTIHKIKHHEVSRSKIYIEMACGDHLVVNN
SRSSRTARALRHHKYRKTCKRCRVSDDLNKFLTKANEDQTSVKVKVVSAP
TRTKKAMPKSVARAPKPLENTEAAQAQPSGSKFSPAIPVSTQESVSVPAS
VSTSISSISTGATAALVKGNTNPITSMSAPVQASAPALTKSQTDRLEVLL
NPKDEISLNSGKPFRELESELLSRRKKDLQQIYAEERENYLGKLEREITR
FFDRGFLEIKSPILIPLEYIERMGIDNDTELSKQIFRVDKNFCLRPMLAP
NLYNYLRKLDRALPDPIKIFEIGPCYRKESDGKEHLEEFTLNFCQMGSGC
TRENLESIITDFLNHLGIDFKIVGDSCMVYGDTLDVMHGDLELSSAVVGP
IPLDREWGIDKPWIGAGFGLERLLKVKHFKNIKRAARSESYYNGISTNL,

2. Methanosarcina acetivorans C2A, gi|19913912|gb|AAM03608.1|,

SEQ ID NO: 8

MDKKPLDVLISATGLGMSRTGTLHKIKHHEVSRSKIYIEMACGDHLVVNN
SRSCRTARAFRHHKYRKTCKRCRVSDEDINNFLTRSTESKNSVKVRVVSA
PKVKKAMPKSVSRAPKPLENSVSAKASTNTSRSVPSPAKSTPNSSVPASA
PAPSLTRSQLDRVEALLSPEDKISLNMAKPFRELEPELVTRRKNDFQRLY
TNDREDYLGKLERDITKFFVDRGFLEIKSPILIPAEYVERMGINNDTELS
KQIFRVDKNLCLRPMLAPTLYNYLRKLDRILPGPIKVFEVGPCYRKESDG
KEHLEEFTMVNFCQMGSGCTRENLEALIKEFLDYLEIDFEIVGDSCMVYG
DTLDIMHGDLELSSAVVGPVSLDREWGIDKPWIGAGFGLERLLKVMHGFK
NIKRASRSESYYNGISTNL,

3. Methanosarcina barkeri MS, gi|21322023|gb|AAL40867.1|,

SEQ ID NO: 9

MDKKPLDTLISATGLWMSRTGMIHKIKHHEVSRSKIYIEMACGERLVVNN
SRSSRTARALRHHKYRKTCRHCRVSDEDINNFLTKTSEEKTTVKVKVVSA
PRVRKAMPKSVARAPKPLEATAQVPLSGSKPAPATPVSAPAQAPAPSTGS
ASATSASAQRMANSAAAPAAPVPTSAPALTKGQLDRLEGLLSPKDEISLD
SEKPFRELESELLSRRKKDLKRIYAEERENYLGKLEREITKFFVDRGFLE
IKSPILIPAEYVERMGINSDTELSKQVFRIDKNFCLRPMLAPNLYNYLRK
LDRALPDPIKIFEIGPCYRKESDGKEHLEEFTMLNFCQMGSGCTRENLEA
IITEFLNHLGIDFEIIGDSCMVYGNTLDVMHDDLELSSAVVGPVPLDREW
GIDKPWIGAGFGLERLLKVMHGFKNIKRAARSESYYNL,

4. Methanosarcina barkeri Fusaro, gi|68081579|gb|EAM92857.1|,

SEQ ID NO: 10

MDKKPLDVLISATGLWMSRTGTLHKIKHYEVSRSKIYIEMACGDHLVVNN
SRSCRTARAFRHHKYRKTCKRCRVSDEDINNFLTRSTEGKTSVKVKVVSA
PKVKKAMPKSVSRAPKPLENPVSAKASTDTSRSVPSPAKSTPNSPVPTSA
PAPSLTRSQLDRVEALLSPEDKISLNIAKPFRELESELVTRRKNDFQRLY
TNDREDYLGKLERDITKFFVDRDFLEIKSPILIPAEYVERMGINNDTELS
KQIFRVDKNLCLRPMLAPTLYNYLRKLDRILPDPIKIFEVGPCYRKESDG
KEHLEEFTMVNFCQMGSGCTRENLESLIKEFLDYLEIDFEIVGDSCMVYG
DTLDIMHGDLELSSAVVGPVPLDREWGIDKPWIGAGFGLERLLKVMHGFK
NIKRASRSESYYNGISTNL,

5. Methanococcoides burtonii,gi|168184912|gb|EAM99637.1|,

SEQ ID NO: 11

MEKQLLDVLVELNGVWLSRSGLLHGIRNFEITTKHIHIETDCGARFTVRN
SRSSRSARSLRHNKYRKPCKRCRPADQIDRFVKKTFKEKRQTVSVFSSPK
KHVPKKPKVAVIKSFSISTPSPKEASVSNSIPTPSISVVKDEVKVPEVKY
TPSQIERLKTLMSPDKIPIQDELPEFKVLEKELIQRRRDDLKKMYEEDRE
DRLGKLERDITEFFVDRGFLEIKSPIMIPFEYIERMGIDKDDHLNKQIFR
VDSMCLRPMLAPCLYNYLRKLDKVLPDPIRIFEIGPCYRKESDGSSHLEE
FTMVNFCQMGSGCTRENMEALIDEFLEHLGIEYEIEADNCVYGDTIDIMH
GDLELSSAVVGPIPLDREWGVNKPWMGAGFGLERLLKVRHNYTNIRRASR
SELYYNGINTNL,

Group 2 PylS sequence

PylSc (Desulfitobacterium hafniense), gi|68168348|gb|EAM96284.1|,

SEQ ID NO: 6

MFLTRRDPPLSSFWTKVQYQRLKELNASGEQLEMGFSDALSRDRAFQGIE
HQLMSQGKRHLEQLRTVKHRPALLELEGLAKALHQQGFVQVVTPTIITKS
ALAKMTIGEDHPLFSQVFWLDGKKCLRPMLAPNLYTLWRELERLWDKPIR
IFEIGTCYRKESQGQHLNEFTMLNLTELGTPLEERHQRLEDMARWVLEAA
GIREFELVTESSVVYGDTVDVMKGDLELASGAMGPHFLDEKWEIVDPWVG
LGGLERLLMIREGTQHVQSMARSLSYLDGVRLNIN,

PylSn (Desulfitobacterium hafniense), gi|68168352|gb|EAM96288.1|

MRGVSQASEEKKRYYRKNVDFFNLVEKIKLWPSRSGTLHGIKAMTRRGNT
AEIVTHCNRRFIIYNSKHSRAARWLRKLHFGVCPHCRIPEWKLQKYSSTV
MSQHYGSHL,

The global alignment (FIG. 1) established that 2 domains are present in PylS. The first is a poorly conserved N-terminal domain of variable length and varying somewhat from protein to protein. The 2^{nd} and C-terminal domain is much more highly conserved, both among Group I enzymes, as well as between group I and group II PylS enzymes.

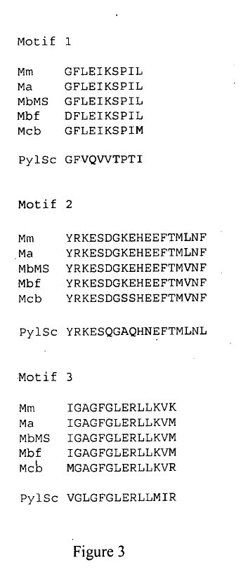

Class II aminoacyl-tRNA synthetase family members are distinguished by three discrete sequences (motifs) that are conserved at low identity (see Srinivasan, G. et al. (2002) Science 296: 1459-1461) within this large family of proteins. These motifs are found in both Group I and Group II PylS enzymes, and are indicated FIG. 2 and FIG. 3. These motifs are involved in the first step of aminoacylation, the adenylylation of the pyrrolysine (see reactions of PylS above). Thus one can identify PylS as a class II protein, and further that this highly conserved domain is the catalytic core of the protein. The three motifs are represented by the following sequences in M. barkeriFusaro and the homologous sequences in the other PylS members can be seen in FIG. 2 (aligned catalytic core) and FIG. 3.

| | |
|---|---|
| Motif 1) 223 DFLEIKSPIL 232, | SEQ ID NO: 15 |
| Motif 2) 294 YRKESDGKEHLEEFTMVN 311, | SEQ ID NO: 16 |
| Motif 3) 383 IGAGFGLERLLKVM 396, | SEQ ID NO: 17 |

The percent identity of the catalytic core of the Group 1 PylS enzymes ranges from 79 to 81% identity against the most diverged group I member (Mcburt) in blastp searches (using the BLOSUM matrix) against each other. Individual sequences in group 1 PylS versus DhPylSc (group 2) have an average 45% identity (64% similarity). The sequences of the catalytic core of the Group I PylS from Methanosarcina mazei, Methanosarcina barkeri MS, Methhanosarcina barkeri Fusaro, Methansosarcina acetivorans, Methanococcoides burtonii, and the Group II DhPylSc were compared to sequence in the non-redundant database at NCBI using a BLASTP search to determine the highest percent identity with a protein that is not a known Group I or Group II PylS. The first hit outside of the group I and Group II PylS catalytic core for the Group I PylS from M. mazei was a threonyl-tRNA synthetase, with 27% identity. The first hit outside of the group I and Group II PylS catalytic core for the Group I Plys from Methanosarcina barkeri MS was gi|42547625|gb|EAA70468.1| (a hypothetical protein from Gibberella zeae PH-1), with 37% identity The first hit outside of the group I and Group II PylS catalytic core for the Group I PylS from Methanosarcina acetivorans was gi|7300003|gb|AAF55175.1| (a hypothetical protein from Drosophila melanogaster), at 28% identity. The first hit outside of the group I and Group II PylS catalytic core for the Group I PylS from M. barkeri Fusaro was gi|42547625|gb|EAA70468.11 (a hypothetical protein FG00875.1 from Gibberella zeae) at 37% identity. The first hit outside of the group I and Group II PylS catalytic core for the Group I PylS from Methanococcoides burtonii was gi|42520791|ref|NP_{—}966706.1| (a threonyl-tRNA synthetase from the Wolbachia endosymbiont of Drosophila melanogaster) at 37% identity. The first hit outside of the group I and Group II PylS catalytic core for Group 2 was gi℄18311304|ref|NP_{—}563238.1| (a threonine-tRNA ligase from Clostridium perfringens str. 13) at 29% identity.

Thus, in addition to polynucleotides encoding proteins comprising the amino acid sequences of the Group I PlyS from the methanogenic Archaea Methanosarcina mazei (Mm), Methanosarcina acetivorans (Ma), Methanosarcina barkeri MS (MbMS), Methanosarcina barkeri Fusaro (MbFus), and Methanococcoides burtonii (Mcburt), or the Group II PylSn and PylSc sequence from the gram positive bacterium Desulfitobacterium haniense, the present method can utilize proteins or polypeptides comprising a sequence that has 79% or more identity with the catalytic core, i.e., those sequences (represented by M. barkeri Fusaro aligning with residues 208-395) in the C terminal domain from the group I Plys from Mm, Ma, MbMS, MbFus, or Mcburt or with the Group II PylSC gene product from Desulfitobacterium hafniense (SEQ ID NO:s 1-6). The present method can also utilize a protein having a similar catalytic core domain with a sequence that is at least 80% identical to the consensus sequence LgkLErditkffvdrgFleiksPilIpaeyverMgInnDteLskQiFrvDknlCLRPMLAPnLYnylRkLdrilpdPlki FEiGpCYRKESdGkeHLeEFTMINfcqmGsgctrenlEalikefLdhlgIdfeivgdscmVYGdTlDvMhgDL ELsSavvGPvpLDreWgidkPWiGaGFGLERLLkv, SEQ ID NO: 13 that was derived from the sequences of the known PlyS enzymes. BlastP analysis of the consensus sequence against the nonredundant database detected Group 1 sequences as the highest identities, returning alignments with the known PylS homologs as: MbFus 97% identical; MbMs 95% identical;Ma 93% identical; Mz 92% identical, and Mcburt 82% identical. Group 2 sequences were clearly next most related protein in database to this consensus sequence heavily weighted for group 1. (DhPylSc: 47% identical.)

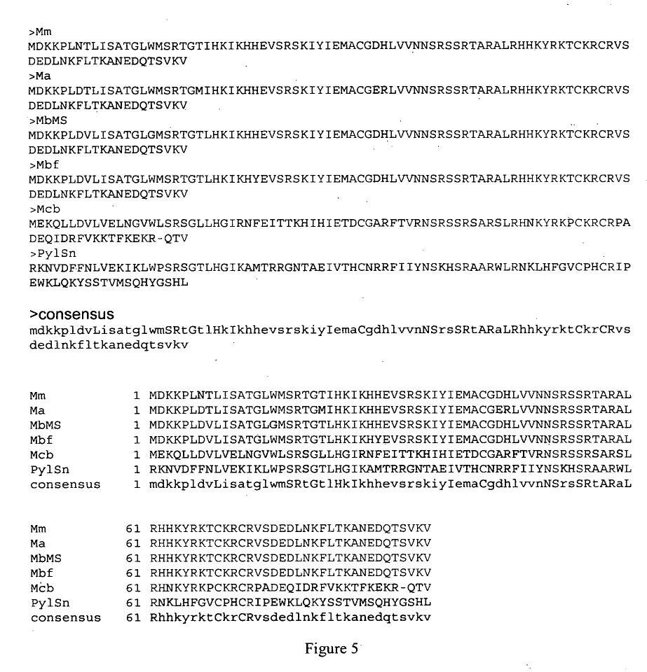

FIG. 5 is an alignment of the conserved regions in the N terminal domain of the pyrrolysyl-tRNA synthetases from the five methanogenic Archaea, SEQ ID NOs. 26-30, a corresponding sequence, SEQ ID NO: 31 from the PlySn gene product of D. hafneinse and a consensus sequence, SEQ ID NO: 25 derived from this alignment. The N terminal conserved regions of the Group 1 PylS enzymes have a percent identity ranging from 46 to 50% and a percent similarity ranging from 72% to 75% against the most diverged group I member (Mcburt) in blastp searches (using the BLOSUM matrix) against each other. Individual sequences in group 1 PylS versus DhPylSc (group 2) have an average 26% identity (38% similarity). The N terminal conserved regions of the Group I PylS enzymes have a percent identity ranging from 50% (Mcb) to 98% (Mbf and MbMS) with the consensus sequence. PylSn has a 35% sequence identity and a 49% sequence similarity with the consensus sequence.

Additionally, while specific reference is made to discrete peptides, polypeptides, and/or proteins, homologs or variants of the disclosed peptides or proteins are specifically contemplated as well. A “variant” as used herein, refers to a peptide or polypeptide whose amino acid sequence is similar to a reference peptide/polypeptide, but does not have 100% identity to the reference peptide/polypeptide sequence. A variant peptide/polypeptide has an altered sequence in which one or more of the amino acids in the reference sequence is deleted or substituted, or one or more amino acids are inserted into the sequence of the reference amino acid sequence, e.g. SEQ ID NO: 1. A variant can have any combination of deletions, substitutions, or insertions. As a result of the alterations, a variant peptide/polypeptide can have an amino acid sequence which is at least about 79, 80, 81, 82, 83, 84, 85, 86, 87, 88, 89, 90, 91, 92, 93, 94, 95, 96, 97, 98, 99, or higher percent, identical to the reference sequence. Variants can be prepared using any suitable method, (e.g., solid phase peptide synthesis, by expression of nucleic acids encoding the variant), and tested for their ability to charge tRNA with lysine These sorts of variants, which may or may not be naturally occurring, are expressly contemplated.

Sequence identity is frequently measured in terms of percentage identity between two aligned sequences. Methods of alignment of sequences for comparison are well-known in the art. Various programs and alignment algorithms are described in: Smith and Waterman, Adv. Appl. Math. 2:482, 1981; Needleman and Wunsch, J. Mol. Bio. 48:443, 1970; Pearson and Lipman, Methods in Molec. Biology 24: 307-331, 1988; Higgins and Sharp, Gene 73:237-244, 1988; Higgins and Sharp, CABIOS 5:151-153, 1989; Corpet et al., Nucleic Acids Research 16:10881-90, 1988; Huang et al., Computer Applications in BioSciences 8:15 5-65,1992; and Pearson et al., Methods in Molecular Biology 24:307-31,1994. Altschul et al. (1994) presents a detailed consideration of sequence alignment methods and homology calculations.

The NCBI Basic Local Alignment Search Tool (BLAST) (Altschul et al., J. Mol. Biol. 215:403-410, 1990) is available from several sources, including the National Center for Biological Information (NBCI, Bethesda, Md.) and on the Internet, for use in connection with the sequence analysis programs blastp, blastn, blastx, tblastn and tblastx. It can be accessed at http://www.ncbi.nlm.nih.gov/BLAST/. A description of how to determine sequence identity using this program is available at http://www.ncbi.nlm.nih.gov/BLAST/blast help.html.

In order to maintain an optimally functional peptide, particular peptide variants will differ by only a small number of amino acids from the peptides disclosed in this specification. Such variants may have deletions (for example of 1, 2 or more amino acid residues), insertions (for example of 1, 2 or more residues), or substitutions that do not interfere with the desired inhibitory activity of the peptides. Substitutional variants are those in which at least one residue in the amino acid sequence has been removed and a different residue inserted in its place. In particular embodiments, such variants will have amino acid substitutions of single residues. Such substitutions generally are made in accordance with the following Table 1 when it is desired to finely modulate the characteristics of the peptide.

TABLE 1

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Greater changes in biological activity may be made by selecting substitutions that are less conservative than those in Table 1, i.e. selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Amino acid sequence variants of a protein can be prepared by any of a variety of methods known to those skilled in the art. For example, random mutagenesis of DNA which encodes a protein or a particular domain or region of a protein can be used, e.g., PCR mutagenesis (using, e.g., reduced Taq polymerase fidelity to introduce random mutations into a cloned fragment of DNA; Leung et al., Bio Technique 1: 11-15 (1989)), or saturation mutagenesis (by, e.g., chemical treatment or irradiation of single-stranded DNA in vitro, and synthesis of a complementary DNA strand; Mayers et al., Science 229: 242 (1985)). Random mutagenesis can also be accomplished by, e.g., degenerate oligonucleotide generation (using, e.g., an automatic DNA synthesizer to chemically synthesize degenerate sequences; Narang, Tetrahedron 39: 3 (1983); Itakura et al., Recombinant DNA, Proc. 3rd Cleveland Sympos. Macromolecules, ed. A. G. Walton, Amsterdam: Elsevier, pp. 273-289 (1981)). Non-random or directed mutagenesis can be used to provide specific sequences or mutations in specific regions. These techniques can be used to create variants which include, e.g., deletions, insertions, or substitutions, of residues of the known amino acid sequence of a protein. The sites for mutation can be modified individually or in series, e.g., by (i) substituting first with conserved amino acids and then with more radical choices depending upon results achieved, (ii) deleting the target residue, (iii) inserting residues of the same or a different class adjacent to the located site, or (iv) combinations of the above. Methods for identifying desirable mutations include, e.g., alanine scanning mutagenesis (Cunningham and Wells, Science 244: 1081-1085 (1989)), oligonucleotide-mediated mutagenesis (Adelman et al., DNA, 2: 183 (1983)); cassette mutagenesis (Wells et al., Gene 34: 315 (1985)), combinatorial mutagenesis, and phage display libraries (Ladner et al., PCT International Appln. No. WO88/06630). The variants can be tested, e.g., for their ability charge tRNA pyrrolysine as described herein

Pyrrolysine Specific tRNA, tRNAPyl.

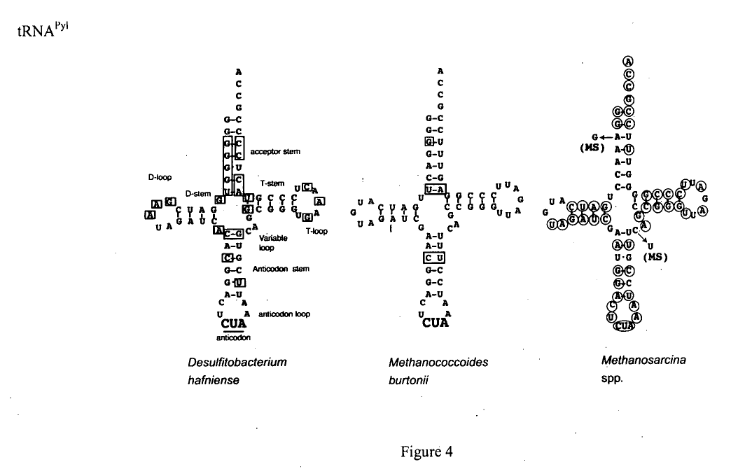

The transfer RNA specific for pyrrolysine, tRNA^{PYL}, also known as tRNA_{CUA}, comprises a CUA anticodon and has a secondary structure with unusual properties as compared to typical tRNAs. (Srinivasan, G. et al. (2002) Science 296: 1459-1461). The secondary structures of three known examples of pylT gene product, tRNA^{Pyl} are shown in FIG. 4. Even though these structures have the expected sizes for the acceptor, D, and T stems and T, and anticodon loops, the anticodon stem of tRNA^{PYL} can form with six, rather than 5 base pairs as shown in FIG. 4. Other unusual features of the known plylT gene products include a small variable loop of 3 base pairs (found in all known examples when folded with a six base pair anticodon stem), a single unpaired base between the acceptor stem and the D-stem, a small D-loop of 5 bases, the extremely unusual CUA anticodon required to decode UAG as pyrrolysine (never found in typical sense tRNA except in mutant amber suppressor tRNA. The conserved bases on tRNA^{Pyl} shown in FIG. 4 allow a consensus sequence to be drawn for tRNA^{Pyl}. The tRNA^{Pyl} consensus sequence is

SEQ ID NO:SEQ ID NO: 14

GGnnnnnnGAUCnnnUAGAUCnnAnGGACUCUAAAUnCnUnnAGnCGGGU
nAnAnUCCCGnnnnUnCCGCCA,.

All known tRNA^{PYL}s have an anticodon loop sequence CUCUAAA. In addition, the D stem of all known tRNA^{PYL}s have in common, the 4 base pairs of the D-stem, the distal 4 base pairs of the T stem, the unpaired bases of the anticodon loop as shown in FIG. 3. The known tRNA^{PYL}s lack the canonical G19 of the D loop found in most tRNA species and canonical C56 of the T loop found in most tRNA species. (Numbering of the bases is in accordance with the universal numbering system for bases in tRNAs of different lengths as described in Sprinzl, M., Horn, C., Brown, M., Ioudovitch, A., and Steinberg, S. (1998) Compilation of tRNA sequences and sequences of tRNA genes. Nucleic Acids Res 26: 148-153.

Based on these findings, it is anticipated that, in addition to the CUA anticodon and the six base pair stem, the tRNA^{PYL}s that can be used in the present methods will have one or more of these unique characteristics. DNA sequences of tRNA^{Pyl} from representative methanogenic Archaea and the gram positive bacterium D. hafniense are shown below.

>DhpylT

SEQ ID NO: 18

GGGGGGTGGATCGAATAGATCACACGGACTCTAAATTCGTGCAGGCGGGT
GAAACTCCCGTACTCCCCGCCA,
>FusaropylT

SEQ ID NO: 19

GGAAACCTGATCATGTAGATCGAAtGGACTCTAAATCCgTTCAGCCGGGT
TAGATTCCCGGGGTTTCCGCCA.
>McburtoniipylT,

SEQ ID NO: 20

GGAGACTTGATCATGTAGATCGAACGGACTCTAAATCCTTTCAGCCGGGT
TAGATTCCCGGAGTTTCCGCCA,
>MapylT,

SEQ ID. NO: 19

GGAAACCTGATCATGTAGATCGAATGGACTCTAAATCCGTTCAGCCGGGT
TAGATTCCCGGGGTTTCCGCCA,
>MmpylT,

SEQ ID NO: 19

GGAAACCTGATCATGTAGATCGaATGGACTCTAAATCCGTTCAGCCGGGT
TAGATTCCCGGGGTTTCCGCCA,
>MSpylT,

SEQ ID NO: 21

gggaacctgatcatgtagatcgaatggactctaaatccgttcagccgggt
tagattcccggggtttccgcca,

Expression Construct

In addition to the polynucleotides encoding the pyyrolysyl-tRNA synthetase, and the tRNA^{PYL}, the expression construct comprises regulatory sequences that are operably linked to such polynucleotides and permit expression of the polynucleotides in a host cell. Thus, the expression construct also comprises a promoter.

As used herein, the term “vector” or “construct” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of the nucleic acids to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

Preferred recombinant expression vectors of the invention comprise a nucleic acid molecule which encodes the tRNA_{CUA} or the pyyroly-tRNA synthetase in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner that allows for expression of the nuleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive or inducible expression of a nucleotide sequence in many types of host cells and those that direct expression of the nucleotide sequence only in certain host cells (e.g., tissue-specific regulatory sequences).

It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides, encoded by nucleic acid molecules as described herein.

The recombinant expression vectors of the invention can be designed for expression of a polypeptide of the invention in prokaryotic or eukaryotic cells, e.g., bacterial cells, such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example, using T7 promoter regulatory sequences and T7 polymerase.

Host Cells

a. The unmodified cells that are modified or transformed in accordance with the present method lack a plyS gene product and/or a plyT gene product. Thus, a host cell can be any prokaryotic cell that is not a methanogenic Arachae or D. hafniense, or any eukaryotic cell. For example, a nucleic acid molecule encoding tRNAcua or pyyroly-tRNA synthetase or both can be expressed in gram negative bacterial cells (e.g., E. coli), insect cells, yeast, or mammalian cells (such as Chinese hamster ovary cells (CHO) or COS cells, human 293T cells, HeLa cells, NIH 3T3 cells, and mouse erythroleukemia (MEL) cells). Other suitable host cells are known to those skilled in the art.
b. Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.
c. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those that confer resistance to drugs, such as G418, hygromycin, or methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene will survive, while the other cells die).
   Method of Making a Protein Comprising a Pyrrolysine or Pyrrolysine Derivative.

A host cell of the invention, such as a prokaryotic or eukaryotic host cell in culture, can be used to produce (i.e., express) a polypeptide or protein that comprises pyrrolysine or a pyrrolysine derivative.

The host cell into which the recombinant expression vectors have been introduced are cultured or maintained in a suitable medium such that tRNA_{CUA} and pyrrolysl-tRNA synthetase are produced. The medium also comprises pyrrolysine or a pyrrolysine derivative such that a polypeptide or protein comprising pyrrolysine or pyrrolysine derivative is produced. Such proteins and polypeptides are encoded by a nucleic acid comprising an internal TAG or UAG codon, i.e. a TAG or UAG codon within the coding sequence.

In one embodiment, proteins comprising a pyrrolysine or pyrrolysine derivative residue are prepared by introducing an expression construct comprising a polynucleotide comprising a protein or polypeptide encoding sequence with an in-frame UAG/UTG codon into a modified/transformed cell of the present invention, exposing the cell to a physiological solution comprising the pyrrolysine or pyrrolysine derivative, and maintaining the cells under conditions which permit expression of the protein or polypeptide encoding sequence. In another embodiment, heterologous proteins comprising a pyrrolysine are prepared by introducing an expression construct comprising a polynucleotide comprising a protein or polypeptide encoding sequence with an in frame UAG codon into an unmodified methanogenic Archaea cell or D. hafniense cell and maintaining the cell under conditions that permit expression of the protein or polypeptide encoding sequence. In those cases, where it is desirable to incorporate a pyrrolysine derivative into such heterologous protein, the cells are also exposed to a physiological solution comprising the pyrrolysine derivative. Proteins comprising a pyrrolysine derivative can also be formed by derivatizing the pyrrolysine residue in a recombinant protein after it is isolated from the present cells as described below.

Kits

Kits for preparing proteins comprising a pyrrolysine or pyrrolysine derivative are also provided. In one embodiment, the kit comprises pyrrolysine and/or one or more pyrrolysine derivatives and expression constructs comprising a polynucleotide encoding a protein with pyrrolysyl-tRNA synthetase activity and a polynucleotide encoding a tRNA^{PYL} In another embodiment, the kit comprises pyrrolysine and/or one or more pyrrolysine derivatives and a transformed cell that expresses a protein with pyrrolysyl-tRNA synthetase activity and a tRNA^{PYL} The kits may also comprise printed instructional materials describing a method for using the reagents to produce such proteins.

Chemical Synthesis of L-pyrrolysine and Derivatives

Provided herein is the chemical synthesis of L-pyrrolysine and its derivatives and attachment of molecules to L-pyrrolysine via addition to its pyrroline ring, or any ring opened variant. According to the procedures described herein, derivatives of L-pyrrolysine may be prepared with one or several of the following alterations:
a. Having altered length of the alkyl chain of the lysine group for all of the derivatives described in b-h.
b. Having an altered linkage (e.g. epsilon nitrogen of lysine and amide of the pyrroline ring) between the functional group (e.g. pyrroline ring) the main chain group (e.g. lysine).
c. Having a pyrroline ring with altered substituents at C2, C3, C4, C5.
d. Having a pyrroline ring with an altered resonance form (e.g. an enamine) with various substituents at C2, C3, C4, C5.
e. Having a pyrroline ring with altered stereochemistry (e.g. (4S,5S))
f. Having a proline ring with various substituents at C2, C3, C4, C5.
g. Having a five-membered group with modified atoms forming the ring (e.g. cyclopentane, furan, thiophene) with various substituents.
h. Having a completely different functional group than a 4-methyl-pyrroline-5 carboxylate (e.g. substituted coumarin, biotin, strepavidin)
i. Having a radioactive element.

Additionally, provided herein are methods for chemical addition of functional groups to L-pyrrolysine following incorporation into recombinant protein. Also included is chemical addition to altered L-pyrrolysine derivatives (e.g. having an enamine) following incorporation into recombinant protein.

The incorporation of L-pyrrolysine and these derivatives into recombinant protein using the expression system described in a separate section of this patent application provides for numerous uses in biotechnology and medicine. These include, but are not exclusive to, the specific labeling the proteins with various tags (fluorescent/FRET, photoactivatible, biotinylated, spin label) for real time fluorescent imaging, single molecule spectroscopy, protein-protein interaction determination, MRI imaging and protein purification.

Chemical synthesis of L-pyrrolysine.

Described herein is the chemical synthesis of L-pyrrolysine shown in the following synthetic scheme and detailed below:

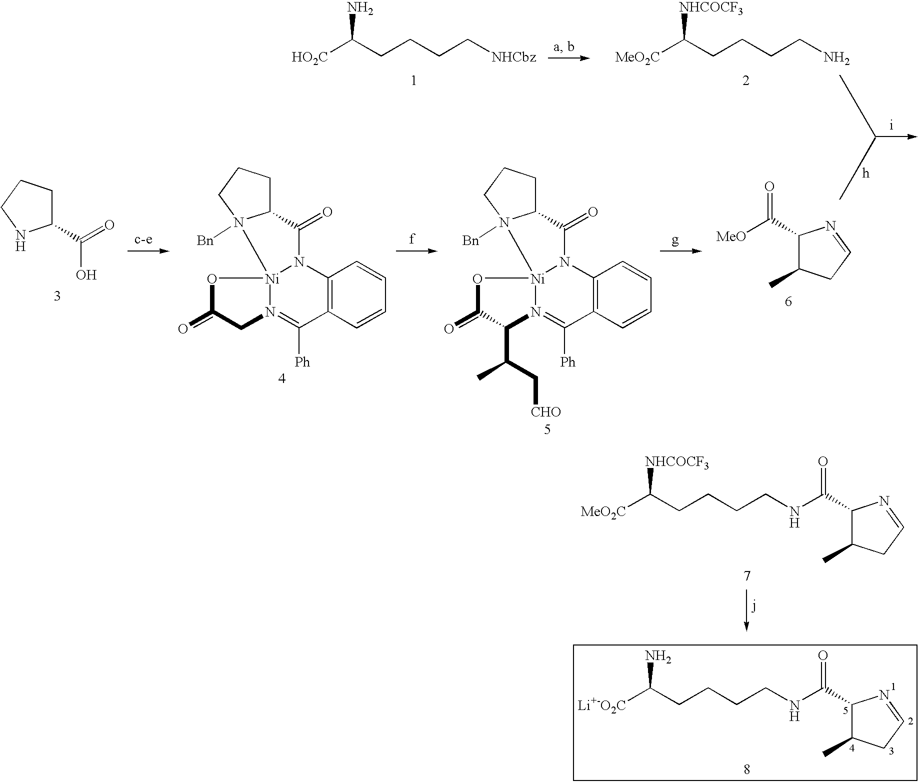

Reagents and conditions: (a) TFAA, Et₃N, CH₂Cl₂ rt, 85%; (b) H₂, Pd/C, MeOH, rt, 99%; (c) ⁱPrOH, KOH, BnCl, rt, 83%; (d) SOCl₂, CH₂Cl₂, then o-aminobenzophenone, rt, 89%; (e) glycine, Ni(NO₃)₂, KOH, MeOH, reflux, 96%; (f) DBU, CH₂Cl₂, crotonaldehyde, rt, 97%; (g) HCl (concd.), MeOH, reflux, then TMSCI, MeOH, rt, 43%; (h) LiOH, THF:H₂O (3:1), rt, ˜100%; (i) 2, DPPA, Et₃N, DMF, rt, 31%; 0) LiOH, THF-MeOH—H₂O (2:2:1), rt, 98%.

Detailed Synthetic Procedure for L-Pyrrolysine

Materials. (R)-N-Benzylproline and (R)-2-[N—(N′-benzylprolyl)amino]benzophenone were prepared by literature methods. (Belokon, Y. N.; Tararov, V. I.; Maleev, V. I.; Savel'eva, T. F.; Ryzhov, M. G. Tetrahedron: Asymmetry 1998, 9, 4249-4252, incorporated herein by reference). Nα-Boc-Nε-Cbz-L-lysine methyl ester was purchased from Aldrich.

Nα-trifluoroacetyl-L-lysine methyl ester (2). To a solution of Nε-Cbz-L-lysine methyl ester (5.8 g, 20 mmol) and Et₃N (5.6 mL, 40 mmol) in CH₂Cl₂ (60 mL) was added trifluoroacetic anhydride (2.8 mL, 20 mmol) at 0° C. The resulting mixture was stirred at rt under N₂ atmosphere for 6 h. The reaction mixture was washed successively with saturated NaHCO₃ and brine, dried over Na2SO4, and evaporated to an oil. Nε-Cbz-Nα-trifluoacetyl-L-lysine methyl ester (6.6 g, 85%) was obtained as an oil after flash chromatography (EtOAc). The product (6.6 g, 17 mmol) was dissolved in MeOH (100 mL) and 10% Pd/C (0.5 g) was added. The mixture was stirred under H2 (1 atm) at rt overnight. The suspension was filtered, and the solvent removed in vacuo to give 4.3 g (99%) of the title compound 2 as a colorless oil. [α]_{D}²⁰=+14.5 (c 0.74 in CHCl₃); ¹H NMR (250 MHz, CDCl₃) δ 1.35-1.51 (m, 2H), 1.59-1.89 (m, 4H), 2.94 (s br, 2H), 3.67 (s, 3H), 4.48 (dd, J=12.6, 7.2 Hz, 1H), 7.57 (d, J=7.4 Hz, 1H), and 8.04 (s br, 2H); 13C{1H} NMR (63 MHz, d₄-methanol) δ 23.9, 27.9, 31.3, 40.5, 53.1, 53.9, 115.6 (q, J=288 Hz), 158.8, and 172.4; MS (ESI) m/z 257 (M⁺+1).

Nickel(II) complex (4). A solution of KOH (15.7 g, 0.28 mol) in MeOH (200 mL) was poured into a stirred mixture of (R)-2-[N—(N′-Benzylprolyl)amino]benzophenone (15.5 g, 40 mmol),⁷ Ni(NO₃)₂-6H₂O (23.3 g, 80 mmol), and glycine (15.0 g, 0.20 mol) in MeOH (200 mL) under an N₂ atmosphere at 40-50° C. The resulting mixture was stirred at 55-65° C. for 1 h, neutralized with AcOH, diluted with water (200 mL), and extracted twice with CH2C12. The organic phase was washed with water and brine, dried (Na₂SO₄), and evaporated to dryness. The residue was subjected to flash chromatography on silica gel (CH₂Cl₂—MeOH=97:3, R_{f}=0.32) to give 19.0 g (96%) of the title compound 4 as a red solid. ¹H NMR (250 MHz, CDCl₃) δ 2.01 (m, 3H), 2.35 (m, 1H), 2.43 (m, 1H), 3.22 (m, 1H), 3.36 (q, J=5.3 Hz, 1H), 3.53 (d, J=12.6 Hz, 1H), 3.61 (m, 2H), 4.36 (d, J=12.6 Hz, 1H), 6.59 (m, 1H), 6.69 (m, 1H), 6.86 (m, 1H) 7.00 (m, 1H), 7.21 (m, 1H), 7.40 (m, 5H), 7.97 (d, J=7.0 Hz, 2H), and 8.17 (d, J=8.7 Hz, 1H); 13C{1H} NMR (63 MHz, CDCl₃) δ 23.5, 32.0, 57.4, 61.1, 63.0, 69.8, 120.7, 124.1, 125.0, 125.5, 126.8, 127.7, 128.3, 129.2, 129.9, 131.6, 132.0, 133.3, 134.5, 142.3, 171.5, 177.1, and 181.2; MS (ESI) m/z498 (M⁺+1).

Michael adduct (5). To a stirring solution of complex 4 (19.0 g, 38 mmol) in CH₂Cl₂ (50 mL) at rt was added DBU (2.9 mL, 19 mmol) followed by crotonaldehyde (2.9 g, 42 mmol). The reaction mixture was stirred for 1 h, and then evaporated to dryness in vacuo. The resulting residue was subjected to flash chromatography on silica gel (CH₂Cl₂-MeOH=95:5, R_{f}=0.36) to give 21.0 g (97%) of the title compound 5 as a deep red solid. ¹H NMR (250 MHz, CDCl₃) δ 1.93 (d, J=6.4 Hz, 3H), 2.07 (m, 2H), 2.26 (d, J=6.6 Hz, 2H), 2.42 (m, 1H), 2.70 (m, 1H), 3.20 (m, 1H), 3.36 (m, 3H), 3.51 (d, J=12.7 Hz, 1H), 3.83 (d, J=3.5 Hz, 1H), 4.33 (d, J=12.7 Hz, 1H), 6.53 (m, 2H), 6.85 (d, J=7.1 Hz, 1H), 7.03 (m, 2H) 7.19 (m, 3H), 7.40 (m, 3H), 7.91 (d, J=7.1 Hz, 2H), 8.17 (d, J=8.6 Hz, 1H), and 9.15 (s, 1H); 13C{1H} NMR (63 MHz, CDCl₃) δ 15.9, 22.9, 30.5, 32.0, 47.1, 56.6, 63.2, 70.2, 72.8, 120.5, 123.2, 126.8, 127.8, 128.7, 128.9, 129.6, 131.4, 131.7, 132.3, 133.1, 133.4, 133.7, 142.4, 171.4, 177.2, 180.1, and 199.9. MS (ESI) m/z 568 (M++1).

(4R,5R)-4-Methyl-1-pyrroline-5-carboxylic acid methyl ester (6). A solution of 5 (9.4 g, 17 mmol) in methanol (100 mL) was added slowly to a stirring solution of concentrated HCl (5 mL) under reflux. After disappearance of the red color of the complex, the reaction mixture was evaporated to dryness under reduced pressure. Methanol (200 mL) and TMSCl (5.3 mL, 42 mmol) were added with stirring. After allowing the reaction mixture to stir overnight at rt under an N2 atmosphere, the solvent was removed in vacuo, and EtOAc (200 mL) was added, followed by saturated NaHCO₃ (100 mL). The organic phase was separated, and the aqueous phase was extracted twice with EtOAc. The combined organic phases were washed with brine, dried (Na₂SO4), and evaporated. The resulting residue was purified by column chromatography on silica gel by eluting with EtOAc-hexane (1:4, R_{f}=0.36) to recover 6.2 g (96%) of (R)-2-[N—(N′-benzylprolyl)amino]benzophenone, and then with EtOAc to give 1.00 g (43%) of title compound 6 as a colorless oil. [α]_{D}²⁰=+7.9 (c=3.2, CHCl₃); ¹H NMR (250 MHz, CDCl₃) δ 1.03 (d, J=6.9 Hz, 3H), 2.06 (ddd, J₁=1.0 Hz, J2=6.2 Hz, J3=18.0 Hz, 1H), 2.36 (m, 1H), 2.76 (dd, J₁=8.8 Hz, J2=18.0 Hz, 1H), 3.61 (s, 3H), 4.41 (dd, J=6.2, 2.0 Hz, 1H), and 7.57 (t, J=1.0 Hz, 1H); 13C{1H} NMR (63 MHz, CDCl₃) δ 20.1, 34.8, 45.8, 52.5, 81.5, 170.0, and 173.1; MS (ESI), m/z 142 (M++1).

Nα-Trifluoroacetyl-L-pyrrolysine methyl ester (7). Ester 6 (1.00 g, 7.1 mmol) and LiOH.H₂O (300 mg, 7.10 mmol) were dissolved in THF—H₂O (3:1, 10 mL). The reaction mixture was tirred at rt for 3 h, and evaporated to dryness under reduced pressure. The resulting product was mixed with Nα-protected lysine 2 (1.8 g, 7.1 mmol), Et₃N (2.0 mL, 14 mmol) and DPPA (2.3 g, 8.5 mmol), and dissolved in DMF (50 mL). After stirring overnight at rt under an N2 atmosphere, EtOAc (100 mL) was added to the mixture. The solution was washed successively with water and brine, dried (Na₂SO₄), and evaporated under reduced pressure. The residue was subjected to flash chromatography on silica gel (EtOAc; R_{f}=0.16) to give 0.80 g (31%) of the title compound 7 as a colorless oil. [α]_{D}²⁰=+12.2 (c=0.74, CHCl₃); ¹H NMR (400 MHz, CD₃OD) δ 1.16 (d, J=6.7 Hz, 3H), 1.25 (m, 2H), 1.43 (m, 2H), 1.77 (m, 2H) 2.10 (dd, J=17.8, 5.0 Hz, 1H), 2.30 (m, 1H), 2.73 (ddd, J=17.8, 6.6, 1.6 Hz, 1H), 3.17 (m, 2H), 3.65 (s, 3H), 3.93 (dd, J=7.4, 2.3 Hz, 1H), 4.38 (dd, J=12.9, 7.4 Hz, 1H), and 7.54 (s, 1H); 13C{1H} NMR (63 MHz, CDCl₃) δ 20.1, 22.0, 29.0, 30.3, 34.7, 37.7, 45.5, 52.6, 81.3, 115.6 (q, J=288 Hz), 129.5, 157.0 (J=38 Hz), 169.7, 171.1, and 173.2; MS (ESI) m/z 366 (M⁺+1).

L-Pyrrolysine lithium salt (8). Compound 7 (120 mg, 0.33 mmol) was dissolved in THFMeOH—H₂O (2:2:1) (5 mL) and then LiOH.H₂O (35 mg, 0.82 mmol) was added. The reaction mixture was stirred at rt for 6 h and then filtered. The filtrate was evaporated under reduced pressure to give a white solid, which was subjected to flash chromatography on silica gel (MeOH-EtOAc=4:1, R_{f}=0.26) to give 0.84 g (98%) of the title compound 8 as a white solid. [α]_{D}²⁰=−2.1 (c=0.25, MeOH); ¹H NMR (400 MHz, CD₃OD) δ 1.17 (d, J=6.8 Hz, 3H), 1.41-1.49 (m, 3H), 1.52-1.61 (m, 3H), 1.76-1.85 (m, 2H), 1.86-1.95 (m, 2H), 2.27 (dddd, J=18.1, 6.6, 1.9, 1.0 Hz, 1H), 2.37 (m, 1H), 2.92 (dd, J=18.1, 8.7, 1H), 3.23 (t, J=6.8 Hz, 3H), 3.52 (t, J=6.1 Hz, 2H), 4.08 (ddd, J=6.3, 4.3, 2.1 Hz, 1H), and 7.74 (s, 1H); 13C{1H} NMR (100 MHz, CD₃OD) δ 20.2, 23.7, 30.2, 32.0, 36.4, 40.1, 56.2, 83.2, 173.1, 174.5, and 174.7; MS (ESI), m/z 262 (M⁺+1).

Nc—Boc-L-lysine Methyl Ester (10). Nα-Boc-Nε-Cbz-L-lysine (3.8 g, 10 mmol) was dissolved in DMF (10 mL) and K2CO3 (2.8 g, 20 mmol) was added at 0° C. The resulting mixture was stirred at 0° C. under N2 atmosphere for 30 min, and MeI (1 mL, 16 mmol) was added dropwise. After stirring overnight at rt under an N2 atmosphere, EtOAc (100 mL) was added, the mixture was washed successively with water and brine, dried (Na₂SO₄), and evaporated under reduced pressure. After flash chromatography on silica gel (EtOAc-hexane=1:3, R_{f}=0.25), Nα-Boc-Nε-Cbz-L-lysine methyl ester (4.00 g, 100%) was obtained as an oil. This oil was dissolved in MeOH (100 mL), 10% Pd/C (0.4 g) was added, and then this mixture was stirred under H2 (1 atm) at room temperature overnight. The suspension was filtered, and the solvent was removed in vacuo to give (88%) 2.3 g of the title compound 10 as a colorless oil. [α]_{D}²⁰=+4.7 (c=5.5, CHCl₃); ¹H NMR (250 MHz, CDCl₃) δ 1.29 (m, 4H), 1.32 (s, 9H), 1.57 (m, 2H), 2.98 (t, J=6.7 Hz, 2H), 3.60 (s, 3H), and 4.13 (m, 1H); 13C{1H} NMR (63 MHz, CDCl₃) δ 22.4, 26.9, 28.2, 31.8, 39.6, 52.3, 53.3, 79.8, 155.5, and 173.2; MS (ESI), m/z 261 (M++1).

Nα-Boc-L-Pyrrolysine methyl ester (11). Ester 6 (0.80 g, 5.7 mmol) and LiOH.H2O (0.26 g, 6.2 mmol) was dissolved in THF-H₂O (3:1, 10 mL). The reaction mixture was stirred at rt for 3 h, and then evaporated to dryness under reduced pressure. The resulting product was mixed with N1-Boc-L-lysine methyl ester 10 (1.5 g, 5.7 mmol), Et₃N (1.2 g, 11.4 mmol) and DPPA (1.8 g, 6.8 mmol), and dissolved in DMF (50 mL). After stirring overnight at rt under an N2 atmosphere, EtOAc (100 mL) was added to the mixture. The solution was washed successively with water and brine, dried (Na₂SO₄), and evaporated under reduced pressure. The residue was subjected to flash chromatography on silica gel (EtOAc) to give 0.66 g (32%) of the title compound 11 as a colorless oil. [α]_{D}²⁰=+8.0 (c=0.89, CHCl₃); ¹H NMR (500 MHz, CDCl₃) δ 1.27 (d, J=6.8 Hz, 3H), 1.30-1.40 (m, 3H), 1.42 (s, 9H), 1.46-1.56 (m, 3H), 1.59-1.67 (m, 3H), 1.75-1.84 (m, 1H) 2.19 (ddd, J=18.1, 7.6, 2.3 Hz, 1H), 2.35-2.44 (m, 1H), 2.83 (dd, J=18.1, 8.9 Hz, 1H), 3.17-3.30 (m, 2H), 3.71 (dd, J=3.1, 0.5 Hz, 3H), 4.04 (ddd, J=7.3, 4.8, 2.5 Hz, 1H), 4.24 (dd, J=12.3, 7.1 Hz, 1H), 5.20 (d, J=8.1 Hz, 1H), and 7.65 (s, 1H); 13C{1H} NMR (63 MHz, CDCl₃) δ 20.8, 22.6, 28.7, 29.5, 32.5, 35.1, 38.9, 46.0, 52.5, 81.8, 155.8, 169.9, 173.0, and 173.6; MS (ESI), m/z 370 (M⁺+1).

NMR H/D Exchange Studies on Compound (7)

Compound 7 (˜4 mg, ˜10 μmol) was dissolved in CD₃OD (0.75 mL), and D₂O (2 drops) was added. The reaction mixture was kept at rt and monitored by NMR (400 MHz). No change was observed over a 3-day period. NaOD (30% w/w in D₂O, 2 drops) was then added, and the solution monitored by NMR. In addition to the chemical shifts associated with the loss of trifluoroacetyl protecting group, the C₅ proton peak (δ=4.1 ppm) was observed to slowly decrease consistent with H/D exchange. After 25 h, the C₅ proton peak completely disappeared.

To rule out the possibility that the loss of the C₅ proton peak was due to decomposition, the resulting mixture was evaporated at rt under reduce pressure, and the residue was redissolved in CH₃OH and allowed to stir for 1 day. The mixture was then evaporated to dryness in vacuo, and redissolved in CD₃OD. The NMR spectrum was quickly taken, and the C₅ proton peak was again observed. As before, the C₅ proton peak slowly disappeared over a 1-day period.

To evaluate the importance of the imine bond on the acidity of the C5 proton, proline was dissolved in 0.75 mL CD₃OD and 0.20 mL D₂O and treated with (30% w/w in D₂O, 0.050 mL). No evidence for H/D exchange of any proton on the carbons of the proline ring was obsered at rt even after a 1 day period.

Stability Studies of Compound (8) to NaOH. NaOH (6 M, 20 μL, 0.12 mmol) was added to a solution of compound 8 (0.15 M, 100 μL, 0.015 mmol). The resulting mixture was allowed to react at rt over 3 days, during which the degradation of compound 8 was monitored by a ninhydrin TLC assay (CHCl₃—MeOH—NH₄OH(aq)=2:2:1). One new band (R_{f}=0.36) was observed with a retention factor consistent with the lysine lithium salt.

Stability Studies of Compound (8) to LiOH. Compound 8 (21.9 mg, 84 μmol) was dissolved in water (2 mL) and LiOH-H₂O (84.5 mg, 2.0 mmol) was added. This mixture was allowed to react at rt. While initially only starting material was observed by TLC (MeOH-EtOAc=4:1, R_{f}=0.26), after 24 h two new bands (R_{f}=0.10, 0.02) appeared. After 5 days, the solution was evaporated to dryness at rt, redissolved in MeOH (0.5 mL), and purified on chromatography silica gel. The more polar band was shown to be lysine lithium salt based on its Rf, MS [ESI, m/z 137 (M++1)], and NMR spectra. The other new band appears to be a mixture of species based on its NMR and MS data.

Stability Studies of Compound (8) to TFA. Compound 8 (21.7 mg, 0.083 mmol) was dissolved in MeOH (2 mL) and TFA (200 μL) was added. After 1 h, one new band (MeOH-EtOAc=2:1; R_{f}=0.22) was observed by TLC. The mixture was allowed to react to completion for 2 days at rt. The resulting mixture was evaporated to dryness, dissolved in MeOH (0.5 mL), and subjected to chromatography on silica gel. The NMR {¹H NMR (250 MHz, d4-MeOH) δ 1.11 (d, J=6.7 Hz, 3H), 1.28-1.56 (m, 5H), 1.72-1.89 (m, 3H), 2.19 (s br, 1H), 2.41 (s br, 1H), 3.65-3.72 (m, 1H) and 5.04 (s br, 1H)} has some features consistent with L-pyrrolysine, although there are differences in the peaks for the pyrroline ring. The peak for the C₂ imine proton is absent, and the peak for the C5 proton is either absent or shifted. These data together with the MS data [(ESI, m/z 256 (M++1)] suggest that the lysine and pyrroline ring remain associated. Two possible degradation pathways consistent with these data are the tautomerization of the (4R,5R)-4-methyl-1-pyrrolidine-5-carboxylate 8 to either (4R,5R)-4-methyl-2-pyrrolidine-5-carboxylate or (3R)-3-methyl-1-pyrrolidine-2-carboxylate. Additional studies will be required, however, to identify the exact product.

Crystallographic Structure Determination of Michael Adduct (5)

The data collection crystal was a red, rectangular plate. Examination of the diffraction pattern on a Nonius Kappa CCD diffractometer indicated an orthorhombic crystal system. All work was done at 200 K using an Oxford Cryosystems Cryostream Cooler. The data collection strategy was set up to measure a quadrant of reciprocal space with a redundancy factor of 3.6, which means that 90% of the reflections were measured at least 3.6 times. A combination of phi and omega scans with a frame width of 1.0 was used. Data integration was done with Denzo, and scaling and merging of the data was done with Scalepack. 1 Merging the data and averaging the symmetry equivalent reflections (but not the Friedel pairs) resulted in an Rint value of 0.049. The teXsan package3 indicated the space group to be P212121, based on the systematic absences.

The structure was solved by the Patterson method in SHELXS-86.4 Full-matrix leastsquares refinements based on F2 were performed in SHELXL-93.5 For the methyl group, the hydrogen atoms were added at calculated positions using a riding model with U(H)=1.5×Ueq(bonded C atom). The torsion angle, which defines the orientation of the methyl group about the C—C bond, was refined. The other hydrogen atoms were included in the model at calculated positions using a riding model with U(H)=1.2×Ueq(attached atom). The aldehyde hydrogen atom was located on a difference electron density map and refined isotropically. The final refinement cycle was based on all 6118 intensities and 357 variables and resulted in agreement factors of R1(F)=0.037 and wR2(F2)=0.073. For the subset of data with I>2σ(I), the R1(F) value is 0.031 for 5534 reflections. The value of the Flack parameter is 0.008(9), which indicates that this is the correct enantiomer.6 The final difference electron density map contains maximum and minimum peak heights of 0.50 and −0.20 eÅ-3. Neutral atom scattering factors were used and include terms for anomalous dispersion.

Chemical synthesis of L-pyrrolysine derivatives having an altered length or substitution of the of the lysine alkyl chain. Examples of altered length, substitution within the chain or on the alkyl chain as depicted below. 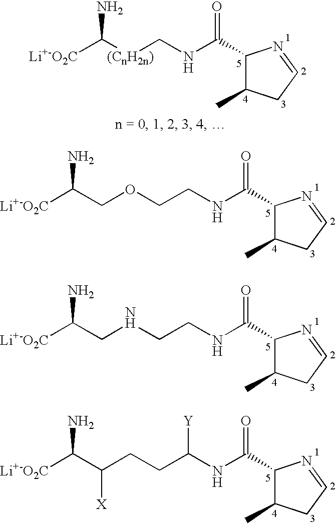
where X or Y can be a proton, any alkyl chain, or alkyl chain with a terminal substitutent such as an hydroxyl, amino, carboxylate groups for attachment of various probes, an azido group for photocrosslinking, or a substituent which introduces a fluorescence or spin tag, or specific binding agents (e.g. biotin).

A procedure for preparing compounds with varied lysine tails (CₙH₂ₙ), n=2,3,4,5 . . . involves simply using a lysine analog with either a shorter or longer alkyl chain (shown in the scheme below).

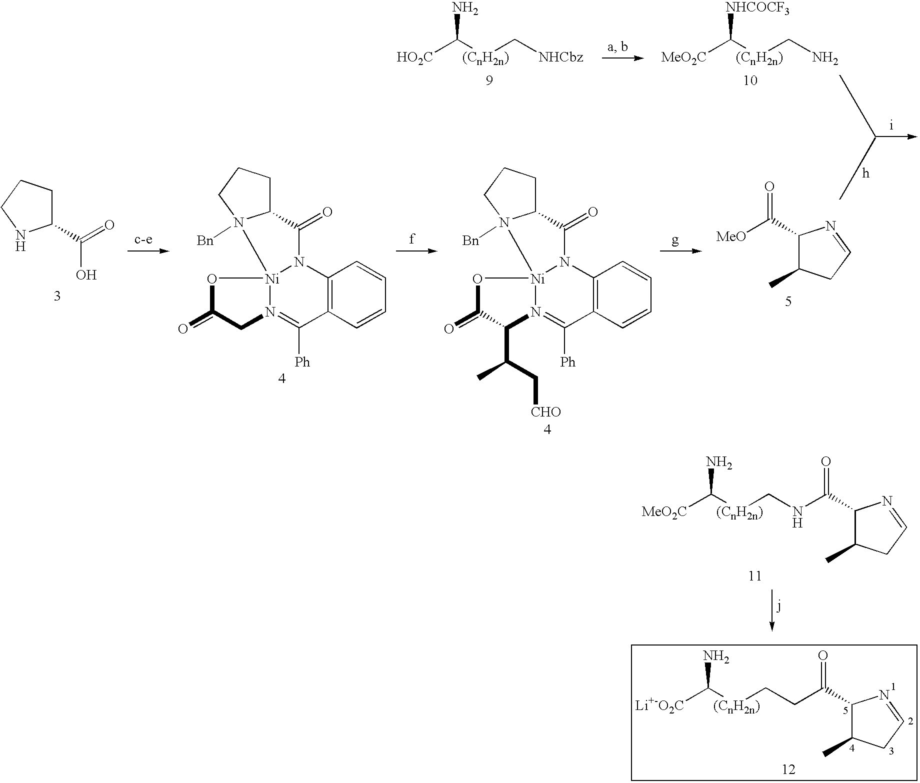

Chemical synthesis of L-pyrrolysine derivatives having an altered linkage (e.g. epsilon nitrogen of lysine and amide of the pyrroline ring) between the functional group (e.g. pyrroline ring) and the main chain group (e.g. lysine). This includes replacing the amide linkage with an ester or ketone, swapping the locations of the carbonyl and nitrogen atoms, or having an ether, amino, thioether linkage.

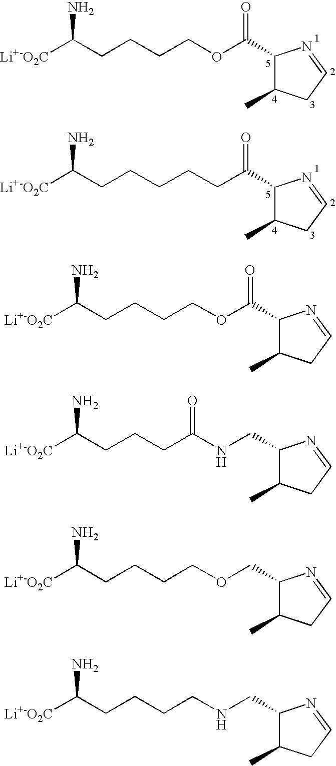

Chemical Synthesis of L-Pyrrolysine Derivatives Having a Pyrroline Ring with Altered Substituents at C2, C3, C4, C5. 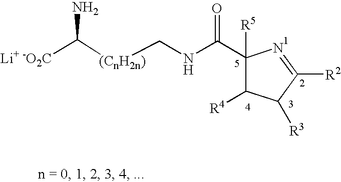
where R2, R3, R4, R5 are either a proton, alkyl chain, halide, hydoxyl, amino, thiol, phosphoryl, particularly those used to link to a fluorescent label (e.g. coumarin), spin label, affinity tag (e.g. biotin, strepavidin), nucleic acid binding group (e.g. adenine, guanine), or photolabile crosslinking group (azido). These labels and tags can also serve as the substituent itself.

General Scheme for the Preparation of Substituted Forms of L-Pyrrolysine

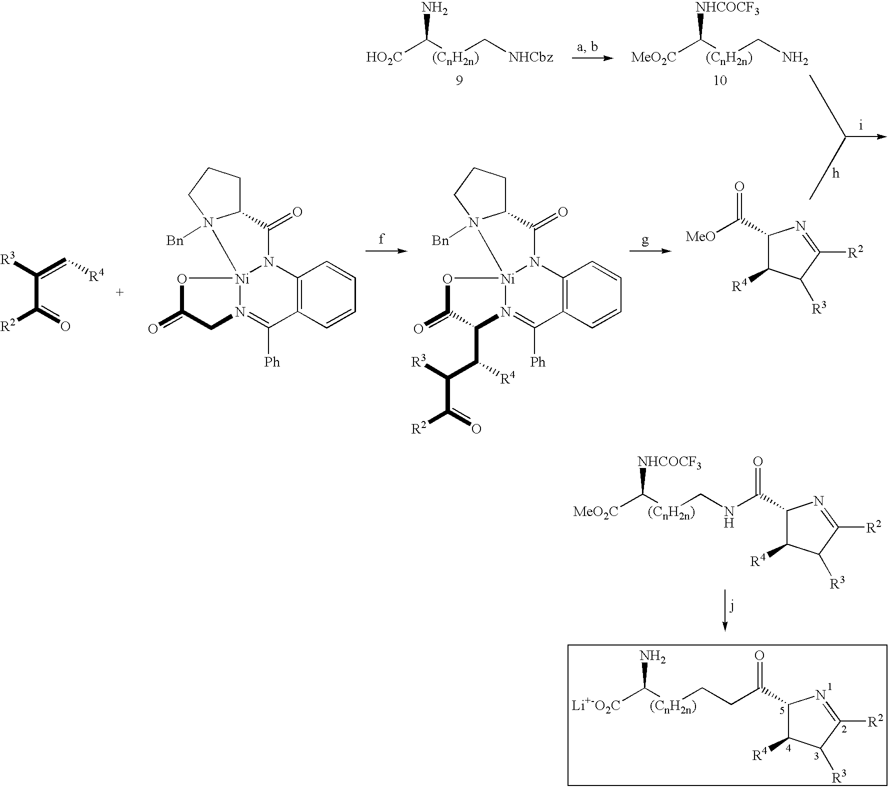

Specific Scheme for Substitution at C2 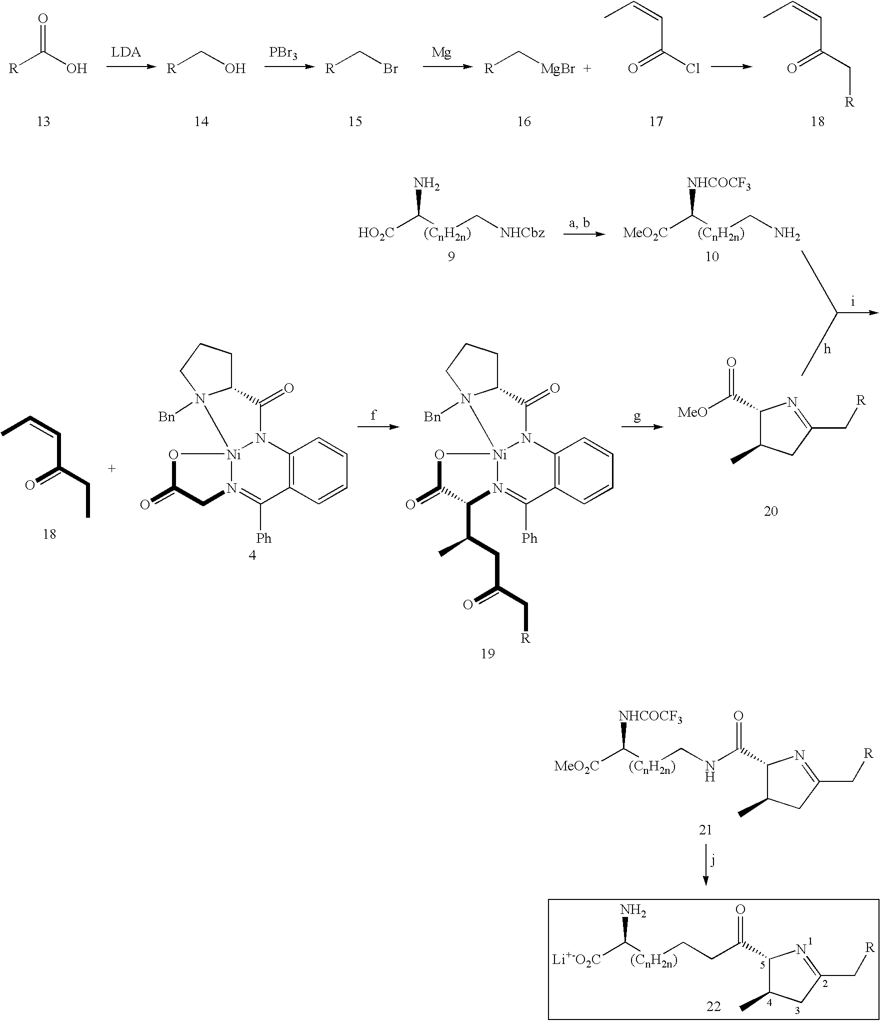
where 13 is one of any number of desired labels or tags containing a free or modified carboxylate side chain that can be modified. Some examples include: 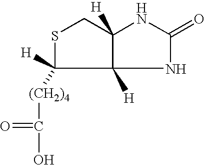
Biotin derivative 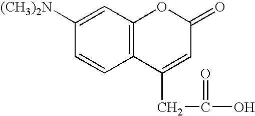
7-dimethylaminocoumarin-4-acetic acid 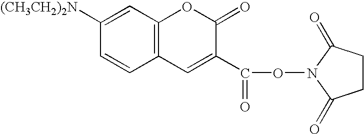
7-hydroxycoumarin-3-carboxylic acid, succinimidyl ester

Chemical Synthesis of L-Pyrrolysine Derivatives Having a Pyrroline Ring with an Altered Resonance form (e.g. an Enamine) with Various Substituents at C2, C3, C4, C5. 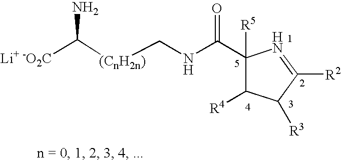
where R², R³, R⁴, R⁵ are either a proton, alkyl chain, halide, hydoxyl, amino, thiol, phosphoryl, particularly those used to link to a fluorescent label (e.g. coumarin), spin label, affinity tag (e.g. biotin, strepavidin), nucleic acid binding group (e.g. adenine, guanine), or photolabile crosslinking group (azido). These labels and tags can also serve as the substituent itself.

L-Pyrrolysine can be converted to its enamine form by treatment with acid.

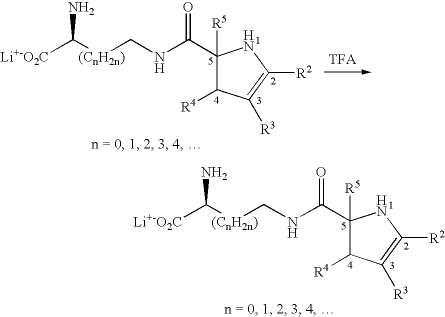

Chemical Synthesis of L-Pyrrolysine Derivatives Having a Pyrroline Ring with Altered Stereochemistry (e.g. (4S,5S)).

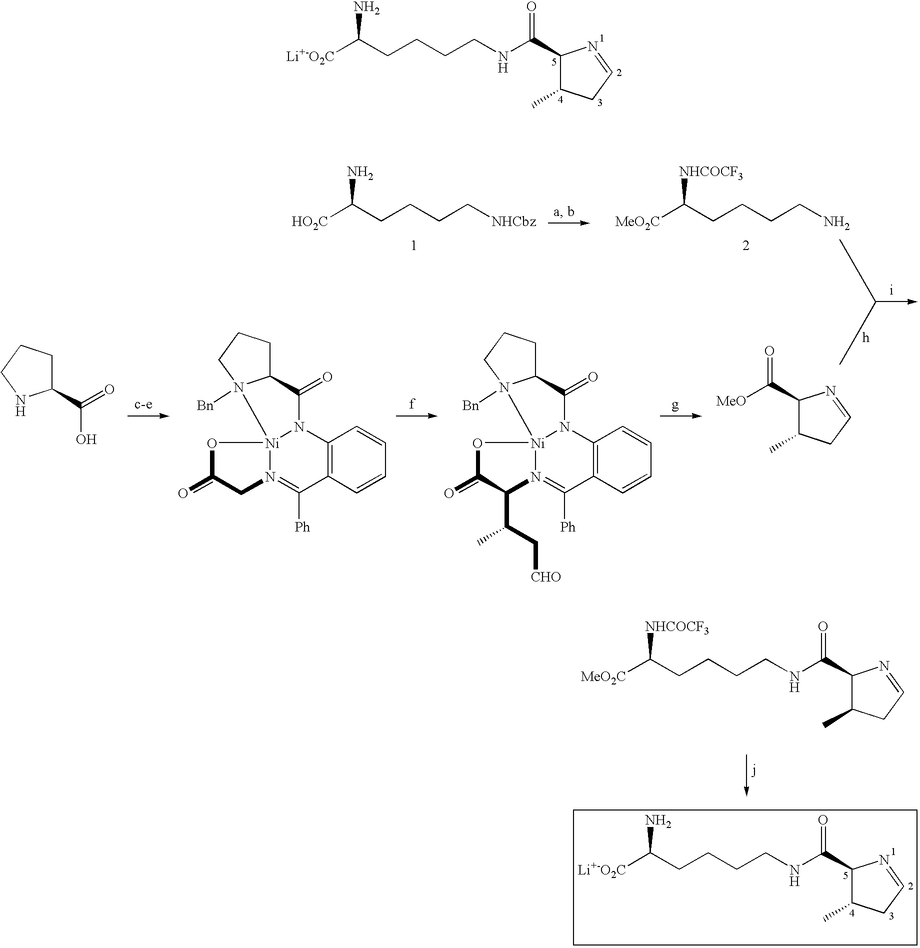

Chemical Synthesis of L-Pyrrolysine Derivatives Having a Proline Ring with Various Substituents at C2, C3, C4, C5.

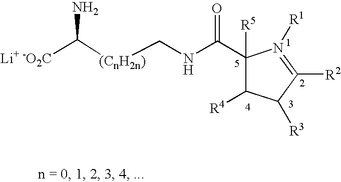

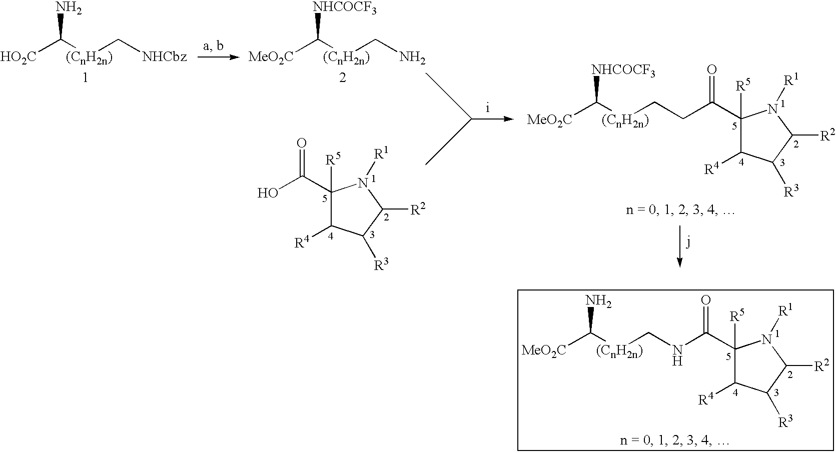

Chemical Synthesis of L-Pyrrolysine Derivatives Having a Five-Membered Group with Modified Atoms Forming the Ring (e.g. Cyclopentane, Hydrofuran, Thiophene) with Various Substituents.

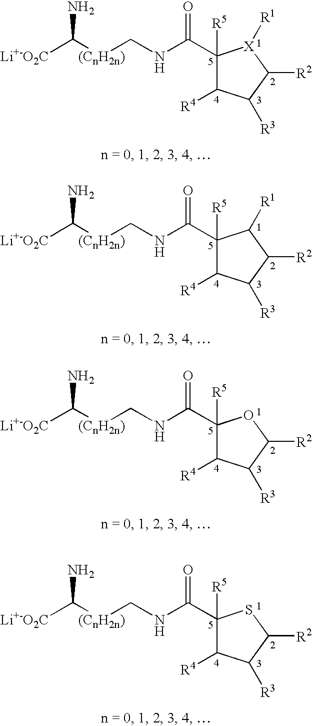

Chemical Synthesis of L-Pyrrolysine Derivatives Having a Completely Different Functional Group than a 4-Methyl-Pyrroline-5 Carboxylate (e.g. Substituted Coumarin, Biotin, Strepavidin).

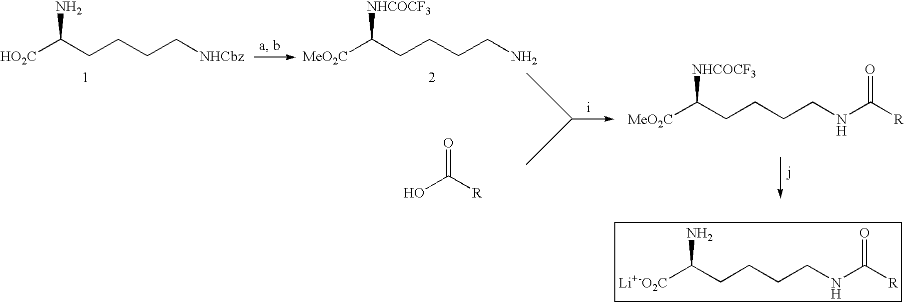

Some examples of potential R—COOH groups include: 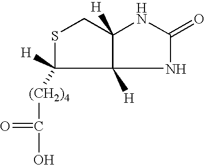
Biotin derivative 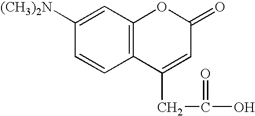
7-dimethylaminocoumarin-4-acetic acid 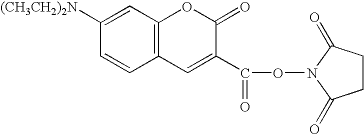
7-hydroxycoumarin-3-carboxylic acid, succinimidyl ester

Chemical synthesis of L-pyrrolysine derivatives containing a radioactive element. Each of the compounds described above can be prepared with a radioactive element (e.g. ¹⁴C, tritium, ³²P, ³⁵S).

(C) Chemical addition of functional groups to L-pyrrolysine following incorporation into recombinant protein.

Crystallographic studies have shown that L-pyrrolysine in proteins can react with various nucleophiles and reducing agents. These reactions lead to the addition of such agents to the C2 carbon of the pyrroline ring. One such example is the reductive addition of dithionite to the imine bond to give a bound sulfate.

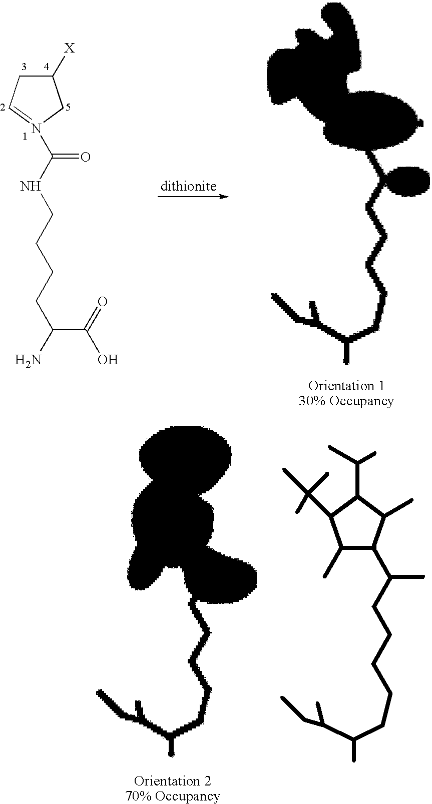

Another is the addition of various substituted hydroxylamines. 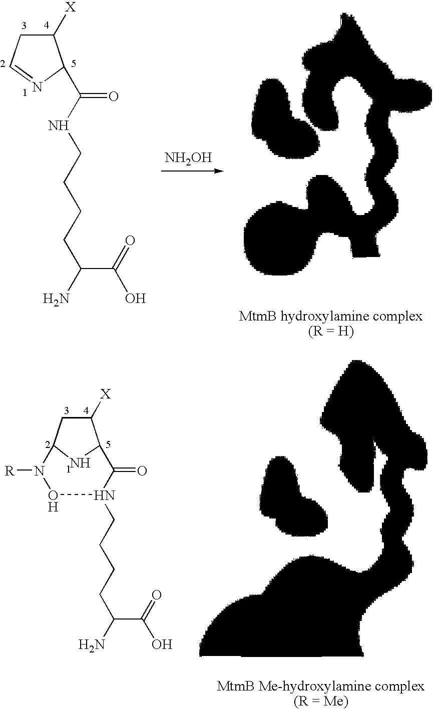
Such additions serve as a potential route for adding various labeling groups including fluorescent labels (e.g. coumarin), spin labels, affinity tags (e.g. biotin, strepavidin), nucleic acid binding groups (e.g. adenine, guanine), photolabile crosslinking groups (azido), or radioactive elements.

Using the expression system described in separate sections of this patent, L-pyrrolysine would be incorporated into recombinant protein. Then in a subsequent step, the L-pyrrolysine would be modified by adding activated forms of the various labeling groups—for instance, a substituted hydroxylamine attached via an alkyl chain to the labeling group.

Chemical addition to altered L-pyrrolysine derivatives (e.g. having an enamine) following incorporation into recombinant protein. Under acidic conditions, we have shown that L-pyrrolysine is converted to its enamine form. The enamine form can be readily alkylated at the C2 position following the Stork enamine reaction. This procedure provides a facile route for the introduction of various substituents.

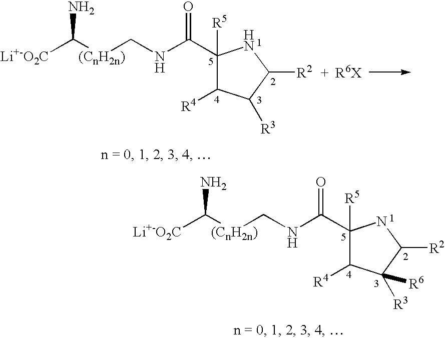

Such additions serve as a potential route for adding various labeling groups including fluorescent labels (e.g. coumarin), spin labels, affinity tags (e.g. biotin, strepavidin), nucleic acid binding groups (e.g. adenine, guanine), photolabile crosslinking groups (azido), or radioactive elements.

Using the expression system described in separate sections of this patent, L-pyrrolysine would be incorporated into recombinant protein. Then in a subsequent step, the L-pyrrolysine would be modified by adding alkyl halide forms of the various labeling groups—for instance, 3-chloromethyl 7-hydroxycoumarin.

Such chloromethylene groups can be easily prepared from their corresponding carboxylates by 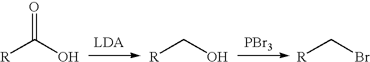
Uses of these compounds

The incorporation of L-pyrrolysine and its modified forms, whether translationally or posttranslationally, has numerous applications in biochemistry, biotechnology, and medicine. Below are listed-some of these technologies, but they are by no means inclusive.

The incorporation of derivatives with photolabile groups enables biochemical and proteomics studies directed at identifying protein protein interactions. A key feature in understanding the biochemical pathways in nature, including those relevant to human disease.

The incorporation of derivatives with radioactive groups serve as enabling technologies for protein detection.

The incorporation of derivatives with fluorescent tags facilitates studies of protein-protein interactions by FRET technologies, single-molecule and intracellular imaging, and the study of protein overexpression.

The incorporation of derivatives with affinity tags provides a means to label proteins with multiple tags and to aid in purification.

The incorporation of derivatives with redox centers provides a means to specifically inject electrons into that center.

### EXAMPLE 1

Most organisms employ UAG as a stop codon, but translation is not terminated at in-frame UAGs in some methyltransferases of methanogenic Archaea. Rather, these codons serve as sense codons and, as determined by crystal structure analyses, UAG encodes pyrrolysine, (4R, 5R)-4-substituted-pyrroline-5-carboxylate, the 22nd amino acid found to be genetically encoded in nature. A key question is whether the UAG-translating tRNAcUA is first charged with lysine and then modified to pyrrolysine for incorporation into the growing polypeptide or whether pyrrolysine is attached as the fully synthesized amino acid to tRNA_{CUA}. Here we show that the latter possibility is feasible by demonstrating the direct pyrrolysylation of tRNA_{CUA} in vitro. This is the first example found in nature of specific aminoacylation of a tRNA with a non-canonical amino acid. The results reported show further that the expression of only two genes, pylT and pylS, that encode tRNA_{CUA} and pyrrolysyl-tRNA synthetase, can expand the genetic code of E. coli to include pyrrolysine. This procedure could potentially be used to immediately expand the genetic code of any species that can incorporate exogenously added pyrrolysine.

The 4-substitutent of pyrrolysine could not be initially assigned, but recent mass spectrometry with MtmB peptide fragments has provided accurate mass measurements indicating that the substituent is a methyl group (K. B. G.-C., Jitesh Soares, Liwen Zhang, Rhonda L. Pitsch, Nanette M. Kleinholz, R. Benjamin Jones, Jeremy J. Wolff, Jon Amster and J.K., unpublished observations). Crystallographic studies also indicated the that most likely substituent at the 4-position of the pyrrolysine ring is a methyl group, and this form of 1-pyrrolysine has been synthesized. Recombinant PylS-His₆ was purified by Ni affinity chromatography from lysates of Escherichia coli expressing the recombinant Methanosarcina barkeri pylS gene modified so as to add a carboxy-terminal hexahistidine tag to the gene product. The tRNA pool extracted from Methanosarcina acetivorans or tRNA_{CUA} transcribed in vitro was used in charging experiments. Charged and uncharged tRNA species were separated by electrophoresis in a denaturing acid-urea polyacrylamide gel and tRNA_{CUA} was specifically detected by northern blotting with an oligonucleotide probe. The oligonucleotide complementary to tRNA_{CUA} could hybridize to a tRNA in the pool of tRNAs isolated from wild-type M. acetivorans but not to the tRNA pool from a pylT deletion mutant of M. acetivorans (A. M., A. Patel, J. Soares, L. R. and J. A. K., unpublished observations).

Both tRNA_{CUA} and aminoacyl-tRNA_{CUA} were detectable in the isolated cellular tRNA pool. Alkaline hydrolysis deacylated the cellular charged species, but subsequent incubation with pyrrolysine, ATP and PylS-His₆ resulted in maximal conversion of 50% of deacylated tRNA_{CUA} to a species that migrated with the same electrophoretic mobility as the aminoacyl-tRNA_{CUA} present in the extracted cellular tRNA pool. The aminoacyl-tRNA_{CUA} synthesized in vitro was also sensitive to mild alkaline hydrolysis. This charged species was not formed by PylS-His₆ in the presence of a mixture of the 20 canonical amino acids each at 50 μM, or only 50 μM lysine, but was formed after the further addition of synthetic pyrrolysine. PylS-His₆ conversion of tRNA_{CUA} to the charged species was therefore dependent on pyrrolysine, even in the presence of other amino acids. To determine whether pyrrolysine itself was present in the cytoplasm, we prepared a cell extract of M. acetivorans and separated the low-molecular-mass metabolite pool from macromolecules by ultrafiltration. The small molecule pool contained a PylS-His₆ substrate for aminoacylation of tRNA_{CUA}. We also demonstrated that PylS-His₆ does not aminoacylate tRNAIYS in the M. acetivorans tRNA pool with either pyrrolysine or lysine. tRNA_{CUA} transcribed in vitro was also aminoacylated with synthetic pyrrolysine by PylS-His₆ in an ATP-dependent reaction. We observed that PylS-His₆ aminoacylated with pyrrolysine a maximum of 43% of tRNA_{CUA} transcribed in vitro during the course of our experiments.

As a prerequisite of tRNA aminoacylation, an aminoacyl-adenylate and pyrophosphate are formed from the amino acid and ATP by an aminoacyl-tRNA synthetase. This reversible activation reaction can be assayed by the isotopic exchange of ³²P-pyrophosphate into ATP dependent on the addition of the cognate amino acid for the aminoacyl-tRNA synthetase in question. PylS-His₆ catalyses a pyrophosphate-ATP isotopic exchange reaction on the addition of synthetic pyrrolysine. This reaction is not dependent on the addition of cellular tRNA. Exchange activity independent of tRNA is typical of a class II aminoacyl-tRNA synthetase. The apparent Kₘ values for pyrrolysine and ATP were 53 μM and 2 μM, respectively. The apparent Vₘₐₓ was 120 nmol min⁻¹ per mg PylS, giving a k_{cat} of 6 min⁻¹ for the exchange reaction. Incubation for as long as 30 min resulted in no detectable isotopic exchange into ATP above background in the presence of a mixture of the canonical 20 amino acids each at 100 μM, or in the presence of 1 mM lysine.

In contrast with the inability of PylS-His₆ to synthesize lysyl-tRNA_{CUA}, we previously observed this activity with amino-terminally His-tagged PylS (His₆-PylS) as assayed by acid precipitation of tRNA ligated to radioactive lysine. However, no lysyl-tRNA synthetase activity was detectable with His₆-PylS by using the gel-shift aminoacylation assay, in agreement with a recent report. In contrast, His₆-PylS does have pyrrolysyl-tRNA synthetase activity as demonstrated by the gel-shift aminoacylation assay. To determine whether PylS lacking either an N-terminal or C-terminal tag sequence acts as a lysyl- or pyrrolysyl-tRNA synthetase, we undertook the following experiments to test whether PylS allows the translation of UAG codons in vivo in E. coli, and whether this would be dependent on the presence of pyrrolysine. As a reporter of UAG translation as a sense codon, we introduced the mtmB1 gene into E. coli BL21 (DE3). The mtmB1 gene encodes the methylamine methyltransferase MtmB, in which pyrrolysine was identified. E. coli BL21 (DE3) expresses recombinant mtmB1 with only trace amounts of the UAG readthrough product, and instead primarily produces a truncated MtmB protein terminating at codon 202, the internal UAG that encodes pyrrolysine in the 452-codon mtmB1 reading frame. Plasmids were constructed bearing combinations of mtmB1, pylS and/or pylT under the control of the T7 promoter, and transformed into E. coli. Expression of these genes was induced in cells growing in the presence and the absence of exogenous 1 mM pyrrolysine. Total cellular proteins from each strain were then separated by SDS-polyacrylamide-gel electrophoresis and mtmB1 gene products were detected by subsequent immunoblotting with polyclonal antibody specific for purified M. barkeri MtmB. All strains produced the amber-truncated product of mtmB1 as a 23-kDa protein; however, the strain expressing pylT, pylS and mtmB1 further expressed large amounts of 50-kDa MtmB, showing UAG readthrough dependent on the presence of pyrrolysine. The pool of amino acids in E. coli did not support the synthesis of an aminoacyl-tRNA_{CUA} that could efficiently translate the UAG codon in mtmB1. The requirement for pyrrolysine could not be replaced by 1 mM lysine or a mixture of the 20 canonical amino acids each at 1 mM. Translation of the mtmB1 UAG codon dependent on pyrrolysine was further dependent on the expression of pylS and pylT, as demonstrated by strains transformed with expression vector containing only pylT or only pylS.

To confirm that synthetic pyrrolysine is incorporated at the UAG-encoded position of mtmB1 by E. coli transformed with pylT and pylS, the insoluble recombinant full-length MtmB protein was partly purified by differential centrifugation and solubilization with urea. After SDS gel electrophoresis, full-length recombinant MtmB was subjected to in-gel digestion with chymotrypsin. An m/z 791.5²⁺ ion was identified, which corresponds to the predicted m/z 791.4²⁺ of the MtmB fragment AGRPGMₒₓGVXGPETSL, (residues 194-208), SEQ ID NO:23 where X is the UAG-encoded residue with the predicted mass of synthetic pyrrolysine. Collision-induced dissociation mass spectrometry confirmed the sequence, and the mass of the UAG-encoded residue was ascertained as 237.2 Da. The predicted molecular mass of the synthetic pyrrolysyl residue is 237.16 Da, thereby confirming that expression of pylT and pylS is sufficient to expand the genetic code of E. coli to include exogenous pyrrolysine. The amino acid might enter the cell because its amide bond allows recognition by a broad-spectrum peptide transporter such as DppA. The synthesis of full-length MtmB further indicates that the E. coli translation factor EF-Tu binds pyrrolysyl-tRNA_{CUA} within a thermodynamic range allowing incorporation into protein during translation.

The current data indicate that pyrrolysine is encoded in DNA using the general mechanism employed for the common set of 20 amino acids. Direct charging of pyrrolysine onto tRNA contrasts with selenocysteine, a genetically encoded non-canonical amino acid synthesized only on tRNA. Several systems have been recently developed to expand and manipulate the genetic code to generate recombinant proteins containing unnatural amino acids. By adding pylS and pylT genes, it should now be possible to generate proteins with the 22nd amino acid incorporated at UAG-targeted sites in any species that can incorporate added pyrrolysine, thereby adding a unique natural amino acid with electrophilic properties.

Methods

Recombinant Proteins

The M. barkeri MS pylS gene was amplified by polymerase chain reaction (PCR) from isolated genomic DNA and cloned into pET 22b (Novagen, Madison, Wis.) to create ppylSH6, which produced PylS with a hexahistidine tag at the C terminus (PylS-His₆) in E. coli BL21 (DE3) (Stratagene, La Jolla, Calif.). PylS-His₆ was isolated from cell extracts in 20 mM sodium phosphate, 500 mM NaCl, 10 mM imidazole pH 7.4, using a Ni-activated trap chelating HP column (Amersham Biosciences Corp., Piscataway, N.J.). PylS-His₆ eluted at 240 mM imidazole during the application of 10-500 mM imidazole in the same buffer to the column. His₆-PylS with a hexahistidine N-terminal tag was used as a partly pure fraction from a nickel-affinity column. Control experiments indicated no pyrrolysyl-tRNA synthetase activity in untransformed E. coli.

The lysS gene was PCR amplified from M. barkeri MS genomic DNA for the recombinant expression of lysS with an N-terminal hexahistidine sequence (His₆-LysS) that eluted at about 130 mM imidazole from the nickel-affinity column.

PyIS substrates

1-Pyrrolysine was synthesized and characterized with the use of ¹³C and ¹H NMR⁹. TLC analysis revealed no other amino acids. The pyrrolysine used in charging experiments was further analysed by electrospray mass spectrometry and revealed two predominant peaks with m/z 256.16 (M+H) and 278.14 (M+Na), where M is 1-pyrrolysine. The cellular tRNA pool was isolated from M. acetivorans C2A (D₆₀₀ 0.6-0.7) growing on trimethylamine at 37° C. in DSM 304 medium because this species is easily lysed. Agarose-gel electrophoresis indicated that 30% of the ethidium bromide staining material in the preparation was tRNA. M. barkeri Fusaro tRNA_{CUA} transcribed in vitro was produced with the DNA template described previously² and the T7-MEGAshortscript transcription kit (Ambion Inc., Austin, Tex.).

The low-molecular-mass cell fraction used in aminoacylation reactions was the supernatant of French-pressed trimethylamine-grown M. acetivorans (27 g in 30 ml 50 mM MOPS pH 7.0) filtered with a 3-kDa Amicon Centricon apparatus (Millipore, Billerica, Mass.), and evaporated to dryness before resuspension in 2 ml doubly distilled water.

Aminoacylation and Pyrophosphate Exchange Assays

The assay for aminoacylation of tRNA_{CUA} (in a volume of 25 μl) contained 0.8-1.7 μM purified PylS-His₆, 50 mM KCl, 1 mM MgCl₂, 5 mM ATP, 0.5 mM dithiothreitol and 50 μM synthetic pyrrolysine in 10 mM HEPES buffer pH 7.2, and 8 μg M. acetivorans tRNA pool preparation or 40 nM of tRNA_{CUA} transcript. The reaction was terminated after 5-30 min at 37° C. with an equal volume of 0.3 M sodium acetate, 8 M urea pH 5.0. Charged and uncharged tRNA were separated by acid-urea acrylamide gel electrophoresis, blotted to nitrocellulose and probed with a 5μ ³²P-end-labelled, 72-base oligonucleotide complementary to tRNA_{CUA}. Radioactivity was analysed with a STORM Phosphorimager (Amersham Biosciences).

The 100-200-μl reactions incubated at 37° C. typically contained 0.3-1 μM PylSHis₆, 10 mM MgCl₂, 25 mM KCl, 1 mM KF, 4 mM dithiothreitol, 2 mM ATP, 100 μM pyrrolysine, 2 mM ³²P-PPᵢ (4-10 d.p.m. pmol⁻¹; PerkinElmer, Boston, Mass.) in 20 mM HEPES-KOH pH 7.2.

Pyrrolysine-Dependent Amber Suppression in E. coli

The M. barkeri MS mtmB1 (GenBank accession number AF013713) was removed with NdeI and EcoRV from plasmid pCJO9, and ligated into MCS2 of pET-Duet to create pEC01 (Novagen). The pylS and pylT genes were PCR amplified from genomic M. acetivorans DNA (GenBank accession number NC 003552) and pylT cloned into the XbaI site directly upstream of MCS1 in pEC01 to create pEC02. The pylS gene was inserted into the NcoI and BamHI sites of MCS1 of pEC02 to create pEC03. The pylTXbaI fragment was excised from pEC03 to create pEC05. All constructs were confirmed by restriction mapping and sequencing. To test for amber suppression, overnight cultures were grown in Luria-Bertani broth (3 ml) with 100 μg ml⁻¹ ampicillin. Subsequently, 200 μl was inoculated into 1 ml fresh medium and grown to a D₆₀₀ of 0.6. The culture (100 μl) was then transferred to a polypropylene tube and induced for 4 h with 1 mM isopropyl μ-d-thiogalactoside in the presence or absence of 1 mM pyrrolysine. The mtmB1 gene products in equivalent amounts of lysates were then analysed by immunoblotting of a SDS 12.5% polyacrylamide gel with affinity-purified rabbit anti-MtmB antibody⁶. The Rainbow Molecular Weight markers (Amersham Biosciences) were used.

To isolate recombinant MtmB for mass spectrometry, E. coli bearing pEC03 (10 ml) was used with 0.75 mM pyrrolysine. The mtmB1 gene products were inclusion bodies. Sequentially washing the pellet from a French-pressed cell lysate with 0, 1, 3, 5 and 7 M urea in 50 mM MOPS pH 7 yielded purified mtmB1 gene products in 7 M urea; these were separated by SDS gel electrophoresis. The 50-kDa MtmB was subjected to in-gel chymotrypsin digestion and peptide sequencing by tandem mass spectrometry.

### EXAMPLE 2

Introduction of a Non-Canonical Amino Acid into a Protein

We describe here a method by which a non-canonical amino acids or its derivatives, that is, those other than the common set of twenty amino acids found in biological organisms, may be introduced into specific positions of proteins using a recombinant organism. The resultant proteins may have various medical, biotechnological, or research applications.

Proteins are produced in living organisms using the information encoded in genes. Decoding the information in genes typically requires a set of twenty aminoacyl-tRNA species that can bring amino acids corresponding to individual codons in genes to the ribosome for polymerization into proteins. Each tRNA species is specific for a particular codon. The tRNA is aminoacylated with one of the twenty canonical amino acids common to all organisms by a dedicated aminoacyl-tRNA synthetase. To this time, only aminoacyl-tRNA synthetases for the common set of twenty amino acids have been discovered in the natural world. We now describe PylS, a new aminoacyl-tRNA synthetase whose substrate for amioacylation is tRNA^{Pyl} (also known as tRNA_{CUA}, the product of the pylT gene) which are highly specific for one another. PylS ligates a newly discovered amino acid, pyrrolysine, to tRNA^{Pyl} with high specificity. It does not detectable utilize other amino acids found in common biological systems to aminoacylate tRNA^{Pyl}. Introduction of the genes encoding PylS and tRNA^{Pyl} into a recombinant organism such as Escherichia coli imparts the ability to decode UAG codons as pyrrolysine, leading the incorporation of this amino acid into recobminant proteins whose encoding genes have been modified to contain a UAG codon. This allows introduction of pyrrolysine into a protein at any place that UAG can be inserted in the corresponding gene. Any pyrrolysine derviative that could be a substrate of naturally occurring or genetically engineered PylS (also known as pyrrolysyl-tRNA synthetase) could be introduced into proteins in a similar manner. This technology makes it possible to insert residues with unique chemical properties into proteins at convenient locations in either a living recombinant system, or an in vitro translation system based on ribosomes from a living system.

PylS is encoded by the pylS gene [Srinivasan, 2002], the sequence of an example can be found in Genbank AY064401 which is from Methanosarcina barkeri DSM 800. Known identiable homologs of this gene can be found in the genomes of Methanosarcina acetivorans, Methanosarcina mazei, Methanosarcina barkeri Fusaro, and Desulfitobacterium hafniense. It is anticipated that others will be discovered as more genomes are sequence which will have properties similar to the described gene and gene product. The pylT gene encoding the UAG decoding tRNA encodes the cognate tRNA of PylS and an example can also be found in Genbank AY064401. Identifiable homologs of pylT can be found in genomes of the same organisms listed above.

Preparation of recombinant proteins. Methods for cloning and handling DNA generally followed those outlined in Sambrook et al. Chromosomal DNA was isolated from M barkeri MS or M acetiovrans C2A as described in Paul et al.

The pylS gene was PCR amplified from the genomic DNA employing primers containing a 5′ NdeI cleavage site (CATATGGATAAAAAACCATTAGATG) SEQ ID NO: ______ and a 3′ Ahol cleavage site (CTCGAGTAGATTGGTTGAAATCCCATTATA) SEQ ID NO: ______. Following purification using the Qiaquick PCR purification kit (Qiagen Inc., Valencia, Calif.) the PCR product was A-tailed using Taq polymerase and ligated at 4° C. overnight into the pGEM-T vector (Promega Inc., Madison, Wis.) to produce pGEMpylS. The pylS gene was then removed from pGEM-TpylS using NdeI and XhoI (Invitrogen Corp., Carlsbad, Calif.) and cloned into pET 22b (Promega) to produce ppylSH6. The pylS gene is modified in this plasmid so that the gene product, PylS-His₆, possesses a hexahistidine tag at the C-terminus. The ppylSH6 plasmid was transformed into E. coli BL21 (DE3) (obtained from Stratagene, La Jolla, Calif.).

Cultures of E. coli transformed with ppylSH6 were grown in Luria-Bertani (LB) broth containing 100 μg/ml ampicillin with shaking at 37° C. for 12-16 hours. A flask containing 500 ml LB broth and 100 μg/ml ampicillin was then inoculated with the overnight culture. The culture was shaken at 37° C. for 1-3 hours until the OD₆₀₀ reached 0.5-0.6. Expression of pylS was then induced by addition of 1 mM isopropyl-beta-D-thiogalactopyranoside (IPTG) followed by 4 hours of further incubation at 37° C. The cells were then centrifuged at 12,000×g at 4° C. and the supernatant discarded. The cell pellet was rinsed in 20 mM sodium phosphate, 500 mM NaCl, 10 mM imidazole, pH 7.4, and then resuspended in 10 mls of the same buffer. The cell suspension was then passed through a French pressure cell at 20,000 psi, and the extract spun at 27,000×g for 20 minutes at 4° C. The supernatant was then used for affinity purification of PylS-His_{6.}

The cell extract (5 mls of 20 mg protein/ml) was loaded at 0.5 ml/min onto a 1 ml HiTrap Chelating HP column (Amersham Biosciences Corp., Piscataway, N.J.) activated with NiSO₄ and pre-equilibrated with the same buffer as the cell extract. The column was washed with 15 mls of the equilibration buffer at 1 mmin, and then a 40 ml gradient of 10 to 500 mM imidazole in equilibration buffer was applied at the same rate. PylS-His₆ eluted as a pure protein at approximately 240 mM imidazole. The purified PylS was then either used immediately or following storage at −20° C. in 40% glycerol.

The lysS gene was PCR amplified from M. barkeri MS genomic DNA with the forward primer being GGAATTCCATATGACNATGGARATHAAYAAY, SEQ ID NO:______ (H=A+T+C) and GCGCCCTCGAGTCARTCYTCYCTYTTCATYTG, SEQ ID NO:______ as the reverse primer. The resultant PCR fragment therefore had a 5′ NdeI and 3′ XhoI site and was ligated with these sites into the expression vector pET15b (Novagen, Inc. Madison, Wis.) similarly digested with NdeI and XhoI to generate plasmid pGS which was used to transform E. coli BL21 (DE3)plysS for expression. This construct produced the lysS gene product with an N-terminal hexahistidine sequence (His₆-LysS).

A cell extract of E. coli transformed with pGS was prepared as described above for nickel affinity chromatography. Five ml cell extract (20 mg protein/ml) was loaded at 0.5 ml/min onto a 1 ml HiTrap Chelating HP column (Amersham Biosciences) activated with NiSO₄ and pre-equilibrated as described above. After loading, the column was washed with 15 ml 50 mM sodium phosphate, 300 mM NaCl, 75 mM imidazole, pH 8.0, at 1 ml/min, followed by a 40 ml gradient of 75 to 500 mM imidazole in the same buffer. His₆-LysS eluted at approximately 130 mM imidazole.

PylS substrates. The methyl variant of pyrrolysine (lysine in amide linkage with (4R, 5R)-4-methyl-pyrroline-5-carboxylate) was synthesized and characterized as described by Hao et al. The NMR and TLC analyses described this paper indicated no contamination by other amino acids. Further analysis of the synthetic pyrrolysine (or pylme) by electrospray mass spectrometry of a 1 mM solution in 50% acetonitrile:water. This was the same preparation used in the charging experiments. Analysis was performed on a Micromass Q-Tof™ II (Micromass, Wythenshawe, UK) mass spectrometer equipped with an orthogonal nanospray source (Z-spray) operated in positive ion mode. Sodium iodide was used for mass calibration. The sample was infused into the electrospray source at a rate of 0.5 ml min⁻¹. Optimal ESI conditions were: capillary voltage 3000 V, source temperature 110° C. and a cone voltage of 60 V. The ESI gas was nitrogen. Q1 was set to optimally pass ions from m/z 50-2000 and all ions transmitted into the pusher region of the TOF analyzer were scanned over m/z with a 1 s integration time. Data was acquired in continuum mode until acceptable averaged data was obtained.

The cellular tRNA pool was isolated from M. acetivorans C2A cells during mid-exponential phase (OD₆₀₀=0.6 to 0.7) growing at 37° C. on DSM 304 media containing 40 mM trimethylamine following the methods of Polycarpo et al. From 100 to 200 mls of culture were centrifuged anaerobically at 15,300×g for 10 minutes at 4° C. The supernatant was quickly decanted and the pellet washed with 300 μl of cold aerobic solution containing 0.3 M sodium acetate and 10 mM EDTA at pH 4.5. The pellet was then resuspended in 300 μl of the same buffer. An equal volume of cold mixture of 5:1 (v/v) phenol:chloroform buffered at pH 4.5 (Ambion Inc., Austin Tex.) was then added and the solution was vortexed 4 times for 30 seconds each with short incubation on ice between each mixing. Centrifugation of the mixture at 18,000×g for 15 min at 4° C. was followed by a second phenol extraction and centrifugation. The aqueous phase then had 3 volumes of cold 100% ethanol added, followed by centrifugation at 18,000×g for 25 minutes at 4° C. The pellet was resuspended in 60 μl of cold 0.3 M sodium acetate, pH 4.5, followed by addition of 400 μl 100% ethanol and centrifugation as before. The supernatant was discarded and the pellet air dried then resuspended in 40 μl cold 10 mM sodium acetate at pH 4.5, and quantified by A₂₆₀. Integrity was checked by electrophoresis of an aliquot on a 1% agarose gel. As indicated, the tRNA pool was deacylated by addition of equal volume of 100 mM Tris and 100 mM NaCl, pH 9.5, and incubation at 70° C. for 30 minutes. Pooled deacylated tRNA to be used in charging reactions was then desalted in 1 ml prepared Sephadex G-25 columns (Amhersam Biosciences) equilibrated with 10 mM HEPES buffer, pH 7.2.

In order to prepare the low molecular weight metabolite pool containing a PylS substrate for aminoacylation of tRNA_{CUA}, 27.2 g of frozen M. acetivorans cells grown on 40 mM trimethylamine for 7 days were resuspended in a final volume of 30 ml of 50 mM MOPS buffer, pH 7.0. The cells were broken by passage through a French Pressure cell at 20,000 psi, and the lysate centrifuged at 40,000 g for 30 minutes at 4° C. The supernatant was then added to Amicon Centricon apparatus (Millipore, Billerica, Mass.) with a molecular weight cut-off of a 3 kDa. The filtrate was collected and 250 ul aliquots in 1.5 ml polypropylene tubes evaporated to dryness in a centrifuge under vacuum at room temperature. The pellet of each tube was then resuspended in 20 μl H₂0 for addition to tRNA_{CUA} aminoacylation reactions.

In vitro transcribed M. barkeri Fusaro tRNA_{CUA} was produced using the double stranded DNA template described previously by Srinivasan et al. Each oligonucleotide (6 μg/μl) was dissolved in ddH₂O, then 30 μl of each solution was mixed, heated at 85° C. for 20 minutes and cooled to room temperature over one hour. The T7-MEGAshortscript transcription kit (Ambion) was used to generate the transcript. A 20 μl reaction was set up according to the manufacturer's protocol using 7.4 μg of template. An additional 1 μl of concentrated (100 U/μl) T7 polymerase (USB Corp., Cleveland, Ohio) was also added to the reaction. The reaction was allowed to go overnight at 37° C. after which 6 U of DNase I was added per reaction. The reaction was terminated by the addition of 20 μl of formamide gel loading buffer (provided in the kit) and heated at 95° C. for 5 minutes. The sample was then loaded onto an 8 M urea/6% polyacrylamide gel. The gel was run at 200 volts for 3 hours at 4° C. The transcript band was visualized using UV shadowing and cut from the gel, and eluted into 300 mM NaOAc, pH 4.5/1 mM EDTA. The tRNA was purified by extraction with acid phenol, precipitated with ethanol, and resuspended in water. The resulting tRNA was refolded by incubation at 85° C. for 15 minutes and cooled to room temperature over 20 minutes. The tRNA was then passed through a G25 size exclusion column and stored at −70° C. in water until use.

Aminoacylation assay in acid-urea gels. The complete assay for aminoacylation of tRNA_{CUA} (25 μl in volume) contained 0.8 μM to 1.7 μM (as noted) purified PyIS-His₆, 50 mM KCl, 1 mM MgCl₂, 5 mM ATP, 0.5 mM dithiothreitol (DTT), 50 μM synthetic pyrrolysine in 10 mM HEPES buffer, pH 7.2, and 8 μg of M. acetivorans tRNA pool preparation or 1 μM of the tRNA_{CUA} transcript. Following incubation at 37° C. for 5 to 30 minutes, the reaction was terminated by addition of an equal volume of 2× loading dye (0.3 M sodium acetate, 8 M urea, pH 5.0) and charged and uncharged tRNA species analysed by acid-urea acrylamide gel electrophoresis following the methods outlined in Jester et al. Samples were electrophoresed in 14% polyacrylamide gels buffered with a 0.3 M sodium acetate-7 M urea solution, pH 5.0, at 50 volts and 4° C. for 24 hours. The same buffer without urea was used in the electrode chambers, and was replaced with fresh buffer every 6-8 hours. Finished gels were transblotted for 2 hours at 40 volts in 4° C. in a blotting solution of 10 mM Tris-acetate, 5 mM sodium acetate, and 0.5 mM EDTA, pH 8.0. The blot was then cross-linked by ultraviolet radiation (15000 μJ/cm2 per side) then prehybrized in 0.25 M sodium phosphate buffer, pH 7.2, and 5.7% SDS for at least 30 minutes. In order to detect charged and uncharged tRNA_{CUA} a 72 base oligonucleotide complementary to full length of tRNA_{CUA} was labeled with γ-³²P ATP and polynucleotide kinase. The probe was hybridized with the blot overnight at room temperature, then agitated at 22° C. for 5 minute in 10 mM sodium citrate, 150 mM NaCl, 1% SDS, pH 7.0, then agitated with two changes of 5 mM sodium citrate, 75 mM sodium chloride, 1% SDS, pH 7.0, for 5 minutes each. Radioactivity on the blot was imaged and analyzed using a STORM Phosphorimager (Amersham Biosciences).

Pyrrolysine dependent pyrophosphate:ATP exchange. The pyrophosphate exchange reaction was essentially performed following the method of Cole and Schimmel. All reactions were performed in duplicate at 37° C. The 100 to 200 μl reactions contained (unless noted) 0.3 to 1 HM PylSHis₆. 10 mM MgCl₂, 25 mM KCl, 1 mM KF, 4 mM DTT, 2 mM ATP, 100 μM synthetic pyrrolysine, 2 mM ³²P-PPi (PerkinElmer, Boston, Mass.) in 20 mM HEPES-KOH (pH 7.2). The specific activity of the radiolabel was adjusted from 4 to 10 dpm/pmol). The concentration of protein was 0.3 to 1 μM. At specific times, 25 μl aliquots were removed and quenched in 500 μl of a stop solution containing 1.6% (w/v) activated acid-washed charcoal (Sigma Chemical Co., St. Louis, Mo.), 80 mM sodium tetrapyrophosphate, and 3.5% (v/v) perchloric acid. Samples were filtered through Whatman GF/C glass-fiber filters and then the filter was washed three times with 5 ml of 40 mM sodium tetrasodium pyrophosphate solution containing 1.7% perchloric acid (v/v) and once with 5 ml 100% ethanol. Filters were dried at 80° C. for 5 minutes then placed in 20 ml scintillation vials containing 10 ml of Ultima Gold F scintillation cocktail (PerkinElmer), shaken once, and counted in a liquid scintillation analyzer. Identical reactions without amino acid, ATP, or enzyme were also set up as negative controls in duplicate. The dependence of the reaction rate on both pyrrolysine and ATP was verified with two independent experiments performed with two different batches of purified enzyme. One substrate was kept constant at a concentration higher than its respective apparent Kₘ value. The ATP concentration was >10 Kₘ for variation of pyrrolysine concentrations, while the concentration of pylme was maintained at ˜2 Kₘ for variation of ATP (in order to conserve the synthetic pyrrolysine substrate). In each case, the concentration of the second substrate was varied over the range Kₘ/5 to 5⁻⁸ Kₘ and the apparent Kₘ and Vₘₐₓ values were determined using Haanes-Wolf plots.

In vivo amber suppression in E. coli expressing pylT and pylS supplemented with synthetic pyrrolysine. Plasmids were constructed that allowed expression of mtmB1 in E. coli BL21 (DE3) along with pylT and/or pylS based on the pET-Duet Cloning Vector (Novagen, Madison, Wis.). The mtmBI gene was from M. barkeri MS (Genbank accession number AF013713)- and derived from plasmid pCJ09 (8). The mtmB1 gene from pCJ09 was removed by NdeI and EcoRV digestion, and ligated into the NdeI and EcoRV restriction sites of MCS2 of pET-Duet in order to create pECOI. The pylS, and pylT genes were obtained using genomic M. acetivorans DNA (Genbank accession number NC 003552) as template for PCR amplification, which were initially cloned into pGEM-T as described above. The pylT gene was amplified from the genome using CTAGAAAGAGCGTGAATTTTGCCGGAGTTTC, SEQ ID NO: ______, as the forward primer, while the reverse primer was TCTAGATATAGGCTCTGGAAAGTGTTTCCTGAT, SEQ ID NO: ______. The pEC02 plasmid was constructed by cloning the pylT gene into the XbaI site directly upstream of MCS 1 in pEC01. The forward primer used to amplify the pylS gene from genomic DNA was CCATGGATAAAA AACCGCTAGACACTCTGATATCTG while the GGATCCTTACAGGTTTGTGGAAATCCCGTTATA was the reverse primer. The plasmid pEC03 was made by insertion of pylS into the NcoI and BamHI sites of MCS 1 of pECO2. Since the mtmB1 gene has an internal NcoI site, the final cloning step of pEC03 was accomplished via partial digest. All plasmid insertions and modifications were confirmed by restriction mapping and partial sequencing of both ends of the inserts. pET-Duet, pEC01, pEC02, and pEC03 were then transformed into E. coli using standard methods to create 4 different strains.

To test for amber suppression, overnight cultures of a strain bearing one of the plasmids were grown in LB broth (3 mL) with 100 ug/ml final ampicillin. Subsequently, 200 μl of each culture was inoculated into 1 ml of fresh LB medium with ampicillin, and grown to an A₆₀₀ of approximately 0.6. Then 100 μl of each culture was transferred to a polypropylene tube and induced with 1 mM IPTG in the presence and absence of 1 mM synthetic pyrrolysine. The cultures were allowed to grow for 4 hours and cell OD following induction was similar in all strains in the presence and absence of pyrrolysine. The cultures were then centrifuged for 1 minute 16,000×g, and the cell pellets lysed in 125 mM Tris HCl, 4.6% SDS, 20% glycerol and 10% (v/v) β-mercaptoethanol, pH 6.8, at 90 degrees for 10 minutes.

Extracts were electrophoresed in a 12.5% SDS-PAGE gel, then electroblotted overnight at 150 mA onto PVDF membrane (Bio-Rad Labs, Hercules, Calif.). The membrane was then incubated for 1 hour at room temperature with Western Breeze blocking solution (Invitrogen). Affinity purified rabbit antibody raised against purified 50 kDa MtmB was then added at 1:2000 dilution and the blot was shaken for 2 hours. The blot was then washed 3× with 100 ml of 10 mM Tris-HCl, 0.9% NaCl pH 7.4, washing buffer; then goat anti-rabbit conjugated secondary antibody (Amersham Biosciences) was then added at 1:2000 in Western Breeze diluent solution (Invitrogen) and the blot was shaken for 2 hours. The blot was agitated again three times with changes of washing buffer, then developed by addition a 100 ml solution containing 0.04 g 1-chloro-4-naphthol, 50 μl 30% hydrogen peroxide and 17% (v/v) ethanol. The Rainbow Molecular Weight markers (Amersham Biosciences) that were used included myosin (220 kDa), phosphorylase b (97 kDa), bovine servum albumin (66 kDa), ovalbumin (45 kDa), carbonic anhydrase (30 kDa), trypsin inhibitor (20.1 kDa), and lysozyme (14.3 kDa).

Cloning of pylT, pylS, and mtmB1

In order to determine the validity of the two presented models, pylT, pylS, and mtmB1 were expressed in E. coli in varying combinations. The specialized cloning vector pETDuet-1 (Novagen, Cat. No. 71146-3) was used for protein expression. The pETDuet-1 vector contains two multiple cloning sites (MCSs) behind T7lac promoter/operators, as well as the lacI gene for isopropyl- -D-thiogalactopyranoside (IPTG)-based expression control and an ampicillin resistance gene. The vector is derived from pBR322 and is controlled by the ColE1 replicon. The first plasmid created contained the mtmB1 gene from M. acetivorans in the MCS2 of pETDuet between the restriction sites for NdeI and EcoRV. This plasmid was designated as pEC01. The pylT gene was then amplified from M. acetivorans genomic DNA template and inserted into the XbaI site of pEC01, which lies just downstream of the T7 promoter in front of MCS1 but upstream of the ribsome binding site (RBS). This construct was designated as pEC02. Another plasmid, pEC03, was then constructed by taking pEC02 and adding pylS to MCS1, in between the restriction sites for NcoI and BamHI (FIG. 7).

| Plasmid Name | Methanosarcina genes present |
|---|---|
| pEC01 | mtmB1 |
| pEC02 | mtmB1, pylT |
| pEC03 | mtmB1, pylT, pylS |

All cloning steps were verified both by restriction digest patterns and direct sequencing using Novagen's provided sequencing primers. All three plasmids were then transformed into the E. coli protein expression strain BL21(DE3) (Stratagene). The pETDuet vector alone without any insert was also transformed into BL21 (DE3). This strain of E. coli is a k(DE3) lysogen with T7 polymerase under control of the lac operator. It can thus be used for IPTG-inducible expression of proteins behind T7 promoters.

The four E. coli strains containing pETDuet, pEC01, pEC02, pEC03 were grown up in overnight Luria-Bertani broth culture with 100 μg/mL ampicillin. One mL LB-ampicillin subcultures were then made from these overnights and grown to an A₆₀₀ of approximately 0.6. Two 100 μL samples of each strain were then used for induction of protein expression. Each culture received 1 mM final concentration of IPTG; one of each pair also received 1 mM final concentration of synthetic pyrrolysine with a methyl group at the 4-substituted position (pylme), synthesized by the Michael Chan laboratory in Biochemistry/Chemistry. The pEC03 culture induction was run in duplicate.

The cultures were then allowed to grow while shaken at 37° for four hours. Cells were then spun down for 1 minute at 14,000 rpm and resuspended in 10 μL SDS-PAGE loading buffer with 10% beta-mercaptoethanol. The suspension was then heated at 90° for 15 minutes to lyse cells and make protein extracts.

Gel Electrophoresis and Western Blotting

The extracts were electorphoresed on a 12.5% SDS-polyacrylamide gel along with a standard MtmB sample and a protein standard marker. The protein samples were then electroblotted onto polyvinyldiene fluoride (PVDF) membrane (Bio-Rad) overnight at 150 mA. The membrane was blocked with Western Breeze™ blocker solution (Invitrogen), then a primary antibody (affinity-purified anti-MtmB) was added to the solution at 1:2000 dilution and the blot was shaken for 2 hours. The membrane was then washed 3 times in a Tris-NaCl washing buffer; horseradish peroxidase (HRP)-conjugated secondary antibody was then diluted 1:2000 in Western Breeze™ diluent (Invitrogen) and added to the membrane, which was then shaken for an additional 2 hours. After three more washes with Tris-NaCl washing buffer, the blot was developed with a solution of 0.04 g 4-choloro-1-naphthol in 17 mL ethanol diluted to 100 mL with water, plus 50 μL 30% hydrogen peroxide.

To further illustrate the ability of pylT and pylS genes to expand the genetic code of E. coli to include pyrrolysine, we modified the uidA gene from E. coli so that it contained a UAG codon replacing codon AAA286 (encoding lysine) by site directed mutagenesis. This mutant uidA gene produces truncated beta-glucuronidase when UAG is recognized as a stop codon in E. colii, which lacks key active sites, and is therefore inactive.

In order to avoid interference by the residue uidA gene, we employed a strain with most ofuidA deleted, E. coli BW25141(DE3). This strain was transformed with pDLGUS, a variant of the pEC03 plasmid in which the mtmB1 gene was removed and the uidA gene introduced in its place. The E. coli strain did not produce and did not possess any beta-glucuronidase activity. However, the pDLO5 strain did possess 0.044 mmol/min·mg activity in the absence of pyrrolysine. However, when the strain was induced with IPTG in the presence of 1 mM synthetic pyrrolysine, the beta-glucuronidase activity increased approximately 100 fold to 4. umol/min·mg protein. This result confirms the applicability of pyrrolysine incorporation to other proteins, as the UAG site in beta-glucuronidase was chosen at random.

In addition, the system above provides a method by which selection of PylS mutants capable of using an expanded spectrum of pyrrolysine derivatives could be selected. The pylS gene could be mutagenized using standard techniques, such as growth in a mutator strain of E. coli, growth of strains bearing the plasmid in chemical mutagens, or by chemical treatment of E. coli in vitro. A pool of mutated pylS gene could be introduced into a strain of E. coli lacking uidA, and growth could be demanded on a beta-glucuronide substrate as sole energy source in the presence of a pyrrolysine derviative. The selective pressure of this condition would allow only those strains carrying a mutated PylS that could ligate the pyrrolysine derivative to tRNA^{Pyl} to grow. These strains could be selected and the mutant PylS whose affinity had been altered such that pyrrolysine derivative are now substrates could be expressed and utilized to produce proteins possessing the pyrrolysine derviative at desired locations.

A similar approach could be used with other catabolic genes with UAG introduced into them, such as that encoding beta-galactosidase (and selection for growth on lactose), or anabolic genes (such as those for synthesis of a key metabolite) and demanding prototrophy for the product of that anabolic gene.

The PylS containing plasmid is isolated from the surviving clone, i.e, the clone having a selected pylS gene that allowed incorporation of the pyrrolysine gene,by standard mini- or maxiprep proceedures. The nucleic acid in the plasmid is sequenced by automated sequencers in common use relying on dideoxynucleotide incorporation, and the sequence of the mutated pylS gene determined.

Alternatively, one could isoalte the mutant enzyme directly from the overproducing strain and the plasmid transformed into any strain bearing the pylT gene and the target gene with UAg codon into which we wished to incorporate the pyl derivative.

Introduction of non-canonical residues using modified versions of the aminoacyl-tRNA synthetases for the common set of twenty amino acids has been a highly sought after commodity. A major stumbling block in the attainment of this goal has been the lack of a aminoacyl-tRNA synthetase and tRNA pair that do not interfere with normal function of the other aminoacyl-tRNA synthetases and tRNA species. PylS and tRNApyl are highly specific for one another, as we demonstrate above. In addition, the structure of pyrrolysine is much unlike other amino acids, so that other aminoacyl-tRNA synthetases do not utilize pyrrolysine, this is clear from our experiments with E. coli. This will allow a much higher level of specificity of incorporation of pyrrolysine or pyrrolysine derivatives in recombinant proteins than previously obtained. No other aminoacyl-tRNA synthetase has been documented which can incorporate pyrrolysine or derivatives of pyrrolysine into proteins.

One application is the incorporation of pyrrolysine into proteins as a means of expressing naturally pyrrolysine-containing proteins of biomedical or biotechnological interest from eucaryotes or procaryotes. Another is the incorporation of pyrrolysine into proteins which normally lack it, as a means of probing their active sites and other aspect of their function. This would amount to the introduction of what is essentially a modified lysine to the at a desired position.

As pyrrolysine has an electrophilic group in the N of the pyrrolysine ring, this will enable specific mdofication of the pyrrolysine once incorporated into a protein with nucleophilic modifying agents. This in principle could allow the modification of recombinant proteins, specifically at the site of pyrrolysine incorporation that could be chosen at will. One such agent is tritiated borohydride. Borohydride will primarily reduce imine bonds in protein. We have already demonstrated that in a large pyrrolysyl-containing protein labeled with deuterated borohydride that only 2 deuterium atoms are incorporated into the protein, and these are incorporated in the small chymotryptic peptide containing pyrrolysine (see attached data). This method could be used as a ready way to radiolabel proteins with high specific activity tritium. Such a kit would have immediate commercial application in biotechnology.

Native pyrrolysine incorporated into proteins may be modified by other nucleophilic agents allowing specific incorporation of fluoresent tags, epitope tags, or biotin tags. Such agents may include those based on hydroxylamine, acylated halides, or cyanogen bromide.

Incorporation into protein of any derivatives of pyrrolysine that can be ligated to tRNA_{CUA} (that is, tRNA^{Pyl}) by unmutated PylS will be possible. Such derviatives could include sidechains allowing photolabelling (such as azido moieties) which will have utility in cross-linking the pyrrolysyl containing protein to other proteins or nucleic acids. Another example of small changes to pyrrolysine that could be used by PylS are EPR-detectable spin labels (such as nitrosyl moieties) which could be incorporated into proteins in order to detect them by EPR, or to measure distances from the point of derivatized pyrrolysine incorporation to existing paramagnetic groups in the protein under study.

The range of derivatives of pyrrolysine can be readily enhanced by selecting for PylS with mutations by method described above for UAG substitution in a required gene and demanding translation using a derivative of pyrrolysine. Such mutated PylS may be able to achieve ligation of very bulky groups to tRNA^{Pyl}, (also known as tRNA_{CUA}), and thereby incorporation into recombinant protein. Such groups could include simple side chains such as those described above, as well as larger ones such as biotinylated derviatives of lysine or pyrrolysine which would allow ready binding of the protein to avidin for isolation or detection. An additional example would be addition of fluorescent labels, which could be used to visualize the protein of interest, monitor binding to other macromolecules, or could be used in fluoresence energy transfer (FRET) studies to measure distances in macromolecular complexes.

The examples described herein are for illustrative purposes only and are not meant to limit the scope of this invention as set forth in the claims.

The invention claimed is:

1. A synthetic amino acid having the structure: 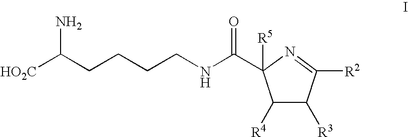
wherein R², R³, R⁴, R⁵ are selected from the group consisting of H, alkyl, halide, hydroxyl, amino, thiol, phosphoryl, azido, alkynyl, substituted alkynyl, vinyl, ketone, aldehyde, carboxylate, ester, a fluorescent label, affinity tag, a nucleic acid binding group, a latent chemical crosslinking group, a photoactivatable crosslinking group and combinations thereof, or a salt thereof.

2. The synthetic amino acid of claim 1 wherein one or more of R², R³, R⁴, and R⁵ comprise a substituent selected from the group consisting of a fluorescence tag, a spin tag, a binding agent, or combinations thereof.

3. The synthetic amino acid of claim 1 wherein the amino acid comprises a radioactive element.

4. The synthetic amino acid of claim 1 wherein the synthetic amino acid is synthetic L-pyrrolysine or a salt thereof.

5. A pyrrolysine derivative of formula II: 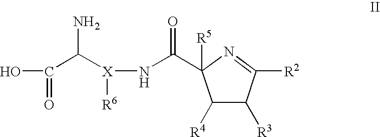
wherein X is selected from the group consisting of (CₙH₂ₙ), n=0 to 6;
wherein R², R³, R⁴, R⁵ are selected from the group consisting of H, alkyl chain, halide, hydroxyl, amino, thiol, phosphoryl, azido, alkynyl, substituted alkynyl, vinyl, ketone, aldehyde, carboxylate, ester, a fluorescent label, affinity tag, a nucleic acid binding group, a latent chemical crosslinking group, a photoactivatable crosslinking group and combinations thereof; and
wherein R⁶ is selected from H, alkyl chain, halide, hydroxyl, amoni, thiol, phosphoryl, azido, alkynyl, substituted alkynyl, vinyl, ketone, aldehyde, carboxylate, ester, a fluorescent label, affinity tag, a nucleic acid binding group, a latent chemical crosslinking group, a photoactivatable crosslinking group and combinations thereof;
or a salt thereof.

6. The derivative of claim 5 wherein the heteroalkyl is selected from the group consisting of ether, thioether, dialkylamine and (CᵥH₂ᵥX′CₙH₂ₙY′CₘH₂ₘ); wherein X′ and Y′ are selected from the group consisting of CR₇R₈, SiR₇R₈, O, S, Se, Te, NR₇, PR₇, AsR₇ and R₇ and R₈ are alkyl or heteroalkyl; and wherein m and n may be the same or different and are 0-6.

7. The derivative of claim 5 wherein X comprises one or more substituents selected from alkyl, or terminal-substituted alkyl, with the terminal substituent selected from azido, fluorescence tag, spin tag, a latent chemical crosslinking group, a photoactivatable crosslinking group, specific binding agent and combinations thereof.

8. The derivative of claim 5 wherein one or more of R¹, R², R³, R⁴, and R⁵ comprise a substituent selected from the group consisting of a fluorescence tag, a spin tag, a binding agent, or combinations thereof.

9. The derivative of claim 5 wherein the amino acid comprises a radioactive element.

10. A pyrrolysine derivative of formula III: 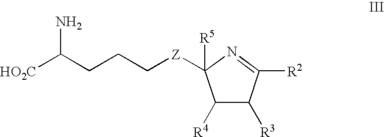
wherein Z is selected from the group consisting of ester, ketone, ether, amido, thioether, and amide; wherein if the linkage is amide, the amide carbonyl and nitrogen are exchanged relative to their positions in pyrrolysine; and
wherein R², R³, R⁴, R⁵ are selected from the group consisting of a proton, alkyl chain, halide, hydroxyl, amino, thiol, phosphoryl, azido, alkynyl, substituted alkynyl, vinyl, ketone, aldehyde, carboxylate, ester, a fluorescent label, affinity tag, a nucleic acid binding group, a latent chemical crosslinking group, a photoactivatable crosslinking group and combinations thereof;
or a salt thereof.

11. The derivative of claim 10 wherein one or more of R¹, R², R³, R⁴, and R⁵ comprise a substituent selected from a fluorescence tag, a spin tag, a binding agent, a latent chemical crosslinking group, a photoactivatable crosslinking group and combinations thereof.

12. The derivative of claim 10 wherein the amino acid comprises a radioactive element.

13. The pyrrolysine derivative of claim 10 selected from the group consisting of 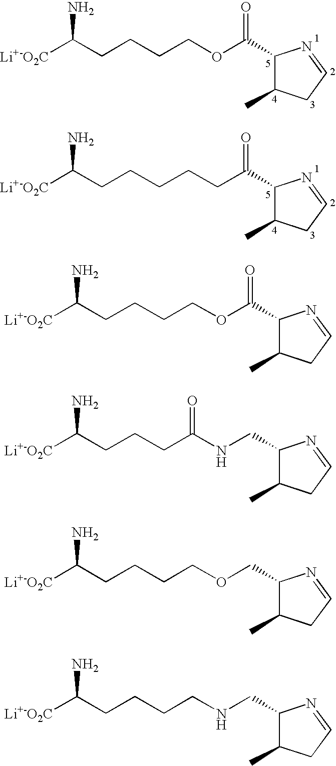
or a salt thereof.

14. The pyrrolysine derivative of claim 13 further comprising one or more substituents on any substitutable C atom on the lysine chain, the substituent selected from alkyl, substituted alkyl, alkynyl, and substituted alkynyl, halide, hydroxyl, amino, thiol, aldehyde, carboxylate, ester, wherein the substituent on the substituted alkyl or alkynyl is selected from azido, fluorescence tag, spin tag, specific binding agent, a latent chemical crosslinking group, a photoactivatable crosslinking group and combinations thereof.

15. The pyrrolysine derivative of claim 13 further comprising one or more substituents on the carbon atoms of the pyrrole ring, the substituents selected from the group consisting of alkyl chain, halide, hydroxyl, amino, thiol, phosphoryl, azido, alkynyl, substituted alkynyl, vinyl, ketone, aldehyde, carboxylate, ester, a fluorescent label, affinity tag, a nucleic acid binding group, a latent chemical crosslinking group, a photoactivatable crosslinking group and combinations thereof.

16. A pyrrolysine derivative of formula IV: 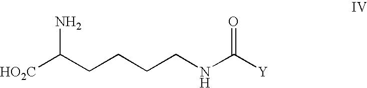
wherein Y is selected from the group consisting of a linear alkyl or heteroalkyl chain, cycloalkyl, heterocycloalkyl, biotin derivative, 7-dimethylaminocoumarin-4-acetic acid and 7-hydroxycoumarin-3-carboxylic acid succinimidyl ester; and
wherein Y is cycloalkyl or heterocycloalkyl the carbon atoms of the cycloalkyl or heterocycloalkyl have substituents selected from the group consisting of H, alkyl, halide, hydroxyl, amino, thiol, phosphoryl, azido, alkynyl, substituted alkynyl, vinyl, ketone, aldehyde, carboxylate, ester, amide, ether, a fluorescent label, a spin label, an affinity tag, a nucleic acid binding group, a latent chemical crosslinking group, a photoactivatable crosslinking group;
or a salt thereof.

17. The derivative of claim 16 wherein Y is selected from the group consisting of 3-membered rings, 4-membered rings, 5-membered rings, and 6-membered rings; and wherein the 3-, 4-, 5-, or 6-membered rings are formed from any combination of C, Si, O, S, Se, N, As, and P atoms; wherein the rings may have one or more unsaturated bonds; and wherein each member of the ring has up to two substituents selected from the group consisting of H, alkyl, halide, hydroxyl, amino, thiol, phosphoryl, a fluorescent label, a spin label, an affinity tag, a nucleic acid binding group, a latent chemical crosslinking group, a photoactivatible crosslinking group, a carbohydrate group, a peptide, or an enzyme inhibitor.

18. The derivative of claim 16 wherein Y is selected from the group consisting of pyrroline, pyrroline in its enamine form, proline, cyclopentane, hydrofuran, and thiophene; and wherein Y comprises substituents selected from the group consisting of H, alkyl, halide, hydroxyl, amino, thiol, phosphoryl, a fluorescent label, a spin label, an affinity tag, a nucleic acid binding group, a latent chemical crosslinking group, a photoactivatible crosslinking group, a carbohydrate group, a peptide, or an enzyme inhibitor.

19. The derivative of claim 16 wherein the amino acid comprises a radioactive element.

20. A method for the synthesis of L-pyrrolysine comprising the steps of:
a) preparing 4-methyl-substituted glutamate γ-semialdehyde
b) cyclizing 4-methyl-substituted glutamate γ-semialdehyde to form the desired pyrrole ring.

21. A method for the synthesis of L-pyrrolysine or a derivative thereof comprising the step of coupling of a carboxylate group or derivative thereof to the epsilon N of lysine or a lysine analog.

22. A method for the chemical addition of functional groups to pyrrolysine or a derivative thereof following incorporation into recombinant protein, the method comprising the steps of:
a) incorporating pyrrolysine or a derivative thereof into a recombinant protein;
b) adding an activated form of a modifying group selected from the group consisting of fluorescent labels, spin labels, affinity tags, nucleic acid binding groups, photolabile group, a latent crosslinking group, a carbohydrate group, a peptide, an enzyme inhibitor, or any group containing a radioactive element.

23. A method for the chemical modification of pyrrolysine or a derivative thereof comprising reacting the pyrrolysine or derivative with a reactive group selected from the group consisting of a reducing group, a nucleophile, an electrophile, and an oxidizing group.


SEQUENCE LISTING (57 sequences)

SEQ ID NO: 1 - protein, 188 aa, Methanosarcina mazei
LGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKV

SEQ ID NO: 2 - protein, 188 aa, Methanosarcina acetivorans
LGKLEREITKFFVDRGFLEIKSPILIPAEYVERMGINSDTELSKQVFRIDKNFCLRPMLAPNLYNYLRKLDRALPDPIKIFEIGPCYRKESDGKEHLEEFTMLNFCQMGSGCTRENLEAIITEFLNHLGIDFEIIGDSCMVYGNTLDVMHDDLELSSAVVGPVPLDREWGIDKPWIGAGFGLERLLKV

SEQ ID NO: 3 - protein, 188 aa, Methanosarcina barkeri
LGKLERDITKFFVDRGFLEIKSPILIPAEYVERMGINNDTELSKQIFRVDKNLCLRPMLAPTLYNYLRKLDRILPGPIKVFEVGPCYRKESDGKEHLEEFTMVNFCQMGSGCTRENLEALIKEFLDYLEIDFEIVGDSCMVYGDTLDIMHGDLELSSAVVGPVSLDREWGIDKPWIGAGFGLERLLKV

SEQ ID NO: 4 - protein, 188 aa, Methanosarcina barkeri
LGKLERDITKFFVDRDFLEIKSPILIPAEYVERMGINNDTELSKQIFRVDKNLCLRPMLAPTLYNYLRKLDRILPDPIKIFEVGPCYRKESDGKEHLEEFTMVNFCQMGSGCTRENLESLIKEFLDYLEIDFEIVGDSCMVYGDTLDIMHGDLELSSAVVGPVPLDREWGIDKPWIGAGFGLERLLKV

SEQ ID NO: 5 - protein, 188 aa, Methanococcoides burtonii
LGKLERDITEFFVDRGFLEIKSPIMIPFEYIERMGIDKDDHLNKQIFRVDESMCLRPMLAPCLYNYLRKLDKVLPDPIRIFEIGPCYRKESDGSSHLEEFTMVNFCQMGSGCTRENMEALIDEFLEHLGIEYEIEADNCMVYGDTIDIMHGDLELSSAVVGPIPLDREWGVNKPWMGAGFGLERLLKV

SEQ ID NO: 6 - protein, 288 aa, Desulfitobacterium hafniense
MFLTRRDPPLSSFWTKVQYQRLKELNASGEQLEMGFSDALSRDRAFQGIEHQLMSQGKRHLEQLRTVKHRPALLELEEGLAKALHQQGFVQVVTPTIITKSALAKMTIGEDHPLFSQVFWLDGKKCLRPMLAPNLYTLWRELERLWDKPIRIFEIGTCYRKESQGAQHLNEFTMLNLTELGTPLEERHQRLEDMARWVLEAAGIREFELVTESSVVYGDTVDVMKGDLELASGAMGPHFLDEKWEIVDPWVGLGFGLERLLMIREGTQHVQSMARSLSYLDGVRLNIN

SEQ ID NO: 7 - protein, 454 aa, Methanosarcina mazei
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 8 - protein, 419 aa, Methanosarcina barkeri
MDKKPLDVLISATGLGMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKVFEVGPCYRKESDGKEHLEEFTMVNFCQMGSGCTRENLEALIKEFLDYLEIDFEIVGDSCMVYGDTLDIMHGDLELSSAVVGPVSLDREWGIDKPWIGAGFGLERLLKVMHGFKNIKRASRSESYYNGISTNL

SEQ ID NO: 9 - protein, 438 aa, Methanosarcina acetivorans
MDKKPLDTLISATGLWMSRTGMIHKIKHHEVSRSKIYIEMACGERLVVNNSRSSRTARALRHHKYRKTCRHCRVSDEDINNFLTKTSEEKTTVKVKVVSAPRVRKAMPKSVARAPKPLEATAQVPLSGSKPAPATPVSAPAQAPAPSTGSASATSASAQRMANSAAAPAAPVPTSAPALTKGQLDRLEGLLSPKDEISLDSEKPFRELESELLSRRKKDLKRIYAEERENYLGKLEREITKFFVDRGFLEIKSPILIPAEYVERMGINSDTELSKQVFRIDKNFCLRPMLAPNLYNYLRKLDRALPDPIKIFEIGPCYRKESDGKEHLEEFTMLNFCQMGSGCTRENLEAIITEFLNHLGIDFEIIGDSCMVYGNTLDVMHDDLELSSAVVGPVPLDREWGIDKPWIGAGFGLERLLKVMHGFKNIKRAARSESYYNL

SEQ ID NO: 10 - protein, 419 aa, Methanosarcina barkeri
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMLAPTLYNYLRKLDRILPDPIKIFEVGPCYRKESDGKEHLEEFTMVNFCQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 11 - protein, 416 aa, Methanococcoides burtonii
MEKQLLDVLVELNGVWLSRSGLLHGIRNFEITTKHIHIETDCGARFTVRNSRSSRSARSLRHNKYRKPCKRCRPADEQIDRFVKKTFKEKRQTVSVFSSPKKHVPKKPKVAVIKSFSISTPSPKEASVSNSIPTPSISVVKDEVKVPEVKYTPSQIERLKTLMSPDDKIPIQDELPEFKVLEKELIQRRRDDLKKMYEEDREDRLGKLERDITEFFVDRGFLEIKSPIMIPFEYIERMGIDKDDHLNKQIFRVDESMCLRPMLAPCLYNYLRKLDKVLPDPIRIFEIGPCYRKESDGSSHLEEFTMVNFCQMGSGCTRENMEALIDEFLEHLGIEYEIEADNCMVYGDTIDIMHGDLELSSAVVGPIPLDREWGVNKPWMGAGFGLERLLKVRHNYTNIRRASRSELYYNGINTNL

SEQ ID NO: 12 - protein, 110 aa, Desulfitobacterium hafniense
MRGVSQASEEKKRYYRKNVDFFNLVEKIKLWPSRSGTLHGIKAMTRRGNTAEIVTHCNRRFIIYNSKHSRAARWLRNKLHFGVCPHCRIPEWKLQKYSSTVMSQHYGSHL

SEQ ID NO: 13 - protein, 188 aa, Artificial Sequence
  Description of Artificial Sequence Synthetic consensus sequence
LGKLERDITKFFVDRGFLEIKSPILIPAEYVERMGINNDTELSKQIFRVDKNLCLRPMLAPNLYNYLRKLDRILPDPIKIFEIGPCYRKESDGKEHLEEFTMLNFCQMGSGCTRENLEALIKEFLDHLGIDFEIVGDSCMVYGDTLDVMHGDLELSSAVVGPVPLDREWGIDKPWIGAGFGLERLLKV

SEQ ID NO: 14 - RNA, 72 nt, Artificial Sequence
  Description of Artificial Sequence Synthetic consensus sequence
ggnnnnnngaucnnnuagaucnnanggacucuaaauncnunnagncgggunananucccgnnnnunccgcca

SEQ ID NO: 15 - protein, 10 aa, Methanosarcina barkeri
DFLEIKSPIL

SEQ ID NO: 16 - protein, 19 aa, Methanosarcina barkeri
YRKESDGKEHLEEFTMVNF

SEQ ID NO: 17 - protein, 14 aa, Methanosarcina barkeri
IGAGFGLERLLKVM

SEQ ID NO: 18 - DNA, 72 bp, Desulfitobacterium hafniense
ggggggtggatcgaatagatcacacggactctaaattcgtgcaggcgggtgaaactcccgtactccccgcca

SEQ ID NO: 19 - DNA, 72 bp, Methanosarcina sp.
ggaaacctgatcatgtagatcgaatggactctaaatccgttcagccgggttagattcccggggtttccgcca

SEQ ID NO: 20 - DNA, 72 bp, Methanococcoides burtonii
ggagacttgatcatgtagatcgaacggactctaaatcctttcagccgggttagattcccggagtttccgcca

SEQ ID NO: 21 - DNA, 72 bp, Methanosarcina barkeri
gggaacctgatcatgtagatcgaatggactctaaatccgttcagccgggttagattcccggggtttccgcca

SEQ ID NO: 22 - protein, 15 aa, Methanosarcina barkeri
  MOD_RES (9): UAG-encoded residue with the predicted mass of synthetic pyrrolysine
AGRPGMGVXGPETSL

SEQ ID NO: 23 - RNA, 7 nt, Artificial Sequence
  Description of Artificial Sequence Synthetic anticodon loop sequence
cucuaaa

SEQ ID NO: 24 - DNA, 25 bp, Artificial Sequence
  Description of Artificial Sequence Synthetic primer
catatggataaaaaaccattagatg

SEQ ID NO: 25 - protein, 95 aa, Artificial Sequence
  Description of Artificial Sequence Synthetic consensus sequence
MDKKPLDVLISATGLWMSRTGTLHKIKHHEVSRSKIYIEMACGDHLVVNNSRSSRTARALRHHKYRKTCKRCRVSDEDINNFLTKTSEDKTSVKV

SEQ ID NO: 26 - protein, 145 aa, Methanosarcina mazei
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQES

SEQ ID NO: 27 - protein, 145 aa, Methanosarcina barkeri
MDKKPLDVLISATGLGMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSS

SEQ ID NO: 28 - protein, 143 aa, Methanosarcina acetivorans
MDKKPLDTLISATGLWMSRTGMIHKIKHHEVSRSKIYIEMACGERLVVNNSRSSRTARALRHHKYRKTCRHCRVSDEDINNFLTKTSEEKTTVKVKVVSAPRVRKAMPKSVARAPKPLEATAQVPLSGSKPAPATPVSAPAQA

SEQ ID NO: 29 - protein, 145 aa, Methanosarcina barkeri
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSP

SEQ ID NO: 30 - protein, 145 aa, Methanococcoides burtonii
MEKQLLDVLVELNGVWLSRSGLLHGIRNFEITTKHIHIETDCGARFTVRNSRSSRSARSLRHNKYRKPCKRCRPADEQIDRFVKKTFKEKRQTVSVFSSPKKHVPKKPKVAVIKSFSISTPSPKEASVSNSIPTPSISVVKDEVK

SEQ ID NO: 31 - protein, 110 aa, Desulfitobacterium hafniense; identical to SEQ ID NO: 12

SEQ ID NO: 32 - DNA, 30 bp, Artificial Sequence
  Description of Artificial Sequence Synthetic primer
ctcgagtagattggttgaaatcccattata

SEQ ID NO: 33 - DNA, 31 bp, Artificial Sequence
  Description of Artificial Sequence Synthetic primer
ggaattccatatgacnatggarathaayaay

SEQ ID NO: 34 - DNA, 32 bp, Artificial Sequence
  Description of Artificial Sequence Synthetic primer
gcgccctcgagtcartcytcyctyttcatytg

SEQ ID NO: 35 - DNA, 31 bp, Artificial Sequence
  Description of Artificial Sequence Synthetic primer
ctagaaagagcgtgaattttgccggagtttc

SEQ ID NO: 36 - DNA, 33 bp, Artificial Sequence
  Description of Artificial Sequence Synthetic primer
tctagatataggctctggaaagtgtttcctgat

SEQ ID NO: 37 - DNA, 36 bp, Artificial Sequence
  Description of Artificial Sequence Synthetic primer
ccatggataaaaaaccgctagacactctgatatctg

SEQ ID NO: 38 - DNA, 33 bp, Artificial Sequence
  Description of Artificial Sequence Synthetic primer
ggatccttacaggtttgtggaaatcccgttata

SEQ ID NO: 39 - protein, 10 aa, Methanosarcina mazei
GFLEIKSPIL

SEQ ID NO: 40 - protein, 10 aa, Methanosarcina acetivorans
GFLEIKSPIL

SEQ ID NO: 41 - protein, 10 aa, Methanosarcina barkeri
GFLEIKSPIL

SEQ ID NO: 42 - protein, 10 aa, Methanococcoides burtonii
GFLEIKSPIM

SEQ ID NO: 43 - protein, 10 aa, Desulfitobacterium hafniense
GFVQVVTPTI

SEQ ID NO: 44 - protein, 18 aa, Methanosarcina mazei
YRKESDGKEHEEFTMLNF

SEQ ID NO: 45 - protein, 18 aa, Methanosarcina acetivorans
YRKESDGKEHEEFTMLNF

SEQ ID NO: 46 - protein, 18 aa, Methanosarcina barkeri
YRKESDGKEHEEFTMVNF

SEQ ID NO: 47 - protein, 18 aa, Methanococcoides burtonii
YRKESDGSSHEEFTMVNF

SEQ ID NO: 48 - protein, 18 aa, Desulfitobacterium hafniense
YRKESQGAQHNEFTMLNL

SEQ ID NO: 49 - protein, 14 aa, Methanosarcina mazei
IGAGFGLERLLKVK

SEQ ID NO: 50 - protein, 14 aa, Methanosarcina acetivorans
IGAGFGLERLLKVM

SEQ ID NO: 51 - protein, 14 aa, Methanosarcina barkeri
IGAGFGLERLLKVM

SEQ ID NO: 52 - protein, 14 aa, Methanococcoides burtonii
MGAGFGLERLLKVR

SEQ ID NO: 53 - protein, 14 aa, Desulfitobacterium hafniense
VGLGFGLERLLMIR

SEQ ID NO: 54 - RNA, 72 nt, Desulfitobacterium hafniense
gggggguggaucgaauagaucacacggacucuaaauucgugcaggcgggugaaacucccguacuccccgcca

SEQ ID NO: 55 - RNA, 72 nt, Methanococcoides burtonii
ggggacuugaucauguagaucgaacggacucuaaauccuuucagccggguuagauucccggaguuuccgcca

SEQ ID NO: 56 - RNA, 72 nt, Methanosarcina sp.
ggaaaccugaucauguagaucgaauggacucuaaauccguucagccggguuagauucccgggguuuccgcca

SEQ ID NO: 57 - protein, 6 aa, Artificial Sequence
  Description of Artificial Sequence Synthetic 6xHis tag
HHHHHH